(19) United States
(12) Patent Application Publication
(10) Pub. No.: US 2025/0213656 A1
(43) Pub. Date: Jul. 3, 2025

(54) Compositions and Methods for Treating Cardiovascular Diseases and Disorders

(71) Applicant: Olfactive Ai, Inc., Mountain View, CA (US)

(72) Inventor: Christopher Hanson, Mountain View, CA (US)

(21) Appl. No.: 19/070,810

(22) Filed: Mar. 5, 2025

Related U.S. Application Data
(63) Continuation-in-part of application No. 18/980,129, filed on Dec. 13, 2024, now Pat. No. 12,290,550.
Application No. 18/980,129 is a continuation-in-part of application No. 18/892,760, filed on Sep. 23, 2024.
Application No. 18/892,760 is a division of application No. 18/615,150, filed on Mar. 25, 2024, now Pat. No. 12,102,664.
Application No. 18/615,150 is a continuation-in-part of application No. 18/430,796, filed on Feb. 2, 2024, now Pat. No. 12,115,134.
(63) Continuation-in-part of application No. 18/958,791, filed on Nov. 25, 2024.
Application No. 18/958,791 is a continuation of application No. 18/811,171, filed on Aug. 21, 2024, now Pat. No. 12,186,299.
Application No. 18/811,171 is a division of application No. 18/615,452, filed on Mar. 25, 2024, now Pat. No. 12,102,611.
(60) Provisional application No. 63/615,100, filed on Dec. 27, 2023.

(51) Int. Cl. A61K 38/26; A61K 31/085; A61K 31/202

(52) U.S. Cl. CPC A61K 38/26; A61K 31/085; A61K 31/202

(57) ABSTRACT

Disclosed herein are compounds and ligands, and compositions formed therewith, that modulate insulin secretion by activating endogenous receptors. Also disclosed herein are methods for using the compositions to treat cardiovascular disorders.

### CROSS-REFERENCES TO RELATED APPLICATIONS

This application is a continuation-in-part of U.S. application Ser. No. 18/980,129, filed on Dec. 13, 2024, which is a continuation-in-part of U.S. application Ser. No. 18/892,760, filed on Sep. 23, 2024, which is a divisional of U.S. application Ser. No. 18/615,150, filed on Mar. 25, 2024, now U.S. Pat. No. 12,102,664, which is a continuation-in-part of U.S. application Ser. No. 18/430,796, now U.S. Pat. No. 12,115,134, filed on Feb. 2, 2024, which claims the benefit of U.S. Provisional Application No. 63/615,100, filed on Dec. 27, 2023.

This application is also a continuation-in-part of U.S. application Ser. No. 18/958,791, filed on Nov. 25, 2024, which is a continuation application of U.S. application Ser. No. 18/811,171, filed on Aug. 21, 2024, now U.S. Pat. No. 12,186,299, which is a divisional application of U.S. application Ser. No. 18/615,452, filed on Mar. 25, 2024, now U.S. Pat. No. 12,102,611.

### FIELD OF THE INVENTION

The present invention relates to compositions and methods for treating physiological disorders. More particularly, the present invention relates to compositions and methods for treating cardiovascular disease and disorders by modulating receptor activity.

### BACKGROUND OF THE INVENTION

As is well established, insulin is a potent anabolic hormone that exerts a variety of effects on many types of cells. Some of the main metabolic actions of insulin are stimulating glucose uptake in skeletal muscles and adipocytes, promoting glycogen synthesis in skeletal muscles, suppressing hepatic glucose production, and inhibiting lipolysis in adipocytes.

In most instances, insulin secretion is induced by pancreatic 3-cells when glucagon-like peptide-1 (GLP-1) binds to and activates GLP-1 receptor proteins on endogenous gastrointestinal (GI) cells, such as enteroendocrine L-cells.

As is also well established, in metabolically impaired patients, abnormal insulin secretion and loss of cellular sensitivity to insulin signaling (insulin resistance) can occur, which principally affects liver, muscle, and adipose cells and is selective for glucose and lipid metabolism. Insulin resistance results in a reduction in insulin-mediated glucose uptake by endogenous cells and further results in compensatory hypersecretion of insulin by pancreatic (β-cells to maintain homeostasis. The hypersecretion of insulin by β-cells typically leads to β-cell exhaustion and dysfunction, and ultimately impairment of insulin production by the β-cells (i.e., abnormal insulin secretion).

As is additionally well established, abnormal insulin secretion and associated insulin resistance are associated with various insulin-resistance-induced physiological disorders; particularly, type 2 diabetes mellitus. The prevalence of type 2 diabetes mellitus continues to rise worldwide as lifestyles associated with low energy expenditure and high caloric intake and, hence, metabolic dysfunction, are increasingly adopted, particularly in lower-income and developing countries. Indeed, it is estimated that the number of cases of type 2 diabetes mellitus is projected to rise from 830 million worldwide to 1.3 billion by 2050.

Recent studies also reflect that there is also a strong correlation between insulin resistance and, hence, hyperglycemia associated therewith, and various cardiovascular disorders, such as atherosclerosis by virtue of vascular endothelial dysfunction, vascular smooth muscle cell (VSMC) over-proliferation and migration, and monocyte/macrophage/foam cell accumulation associated therewith.

Recent studies thus also reflect that there is a strong correlation between abnormal insulin secretion and insulin resistance-associated disorders; particularly, type 2 diabetes mellitus, and various cardiovascular disorders, such as atherosclerosis, diabetic cardiomyopathy (DCM) and hypertension.

Various entities have thus developed pharmaceutically active agents and compositions that are adapted to treat abnormal insulin secretion and associated insulin resistance. In view of the strong correlation between activation of the GLP-1 receptor proteins and insulin secretion, the pharmaceutically active agents and compositions are specifically adapted to activate the GLP-1 receptor proteins on endogenous gastrointestinal (GI) cells.

Such pharmaceutically active agents include semaglutide (Ozempic®, Rybelsus®, Wegovy®), dulaglutide (Trulicity®), exenatide (Bydureon BCise®, Byetta®), and liraglutide (Victoza®, Saxenda®).

The noted pharmaceutically active agents (referred to hereinafter as “GLP-1 analogs”) mimic endogenous GLP-1 and are adapted to activate the GLP-1 receptors on endogenous GI cells and, hence, function as GLP-1 receptor agonists.

Although the GLP-1 analogs can effectively activate GLP-1 receptors on pancreatic β-cells and, hence, can induce insulin secretion and, thereby, effectively treat various physiological disorders, there are several drawbacks and disadvantages associated with administration of the GLP-1 analogs to patients.

A major drawback associated with the administration of the GLP-1 analogs to patients is the high risk of adverse pathological events. One such adverse pathological event is hypoglycemia (i.e., low blood glucose), which can, and often will, present in patients that are also taking or being administered commonly prescribed antidiabetic agents, such as basal insulin and sulfonylureas.

There is also a high risk of induced production of anti-GLP-1 antibodies and binding of endogenous GLP-1 and the GLP-1 analogs to the anti-GLP-1 antibodies, which can, and often will, induce adverse immune responses.

A further major drawback associated with the administration of GLP-1 analogs to individuals are the significant side effects that are often presented by the individuals, including nausea, vomiting, diarrhea, abdominal pain, and constipation.

Since most GLP-1 analogs are administered to patients via a subcutaneous injection, a further drawback associated with GLP-1 analog administration is the pain and discomfort associated with the often-prescribed weekly injections.

Although the GLP-1 analogs developed by Novo Nordisk, which are marketed under the tradename Rybelsus, can also be delivered orally, a significantly greater dose of the Rybelsus GLP-1 analog must be orally administered to an individual to match the pharmacokinetics of the Novo Nordisk injectable GLP-1 analog, which is marketed under the tradename Ozempic, i.e., individuals must be orally administered approximately 100.0 mg/week of the Rybelsus GLP-1 analog to match the efficacy of the typically prescribed 0.5 mg/week of the injectable Ozempic GLP-1 analog.

A further major drawback associated with the administration of GLP-1 analogs to individuals is the cost. Indeed, the costs, at present, for a thirty (30) day supply of Ozempic and Rybelsus are approximately $1000.00 and $1200.00, respectively.

As is also well established, insulin secretion can also be induced by pancreatic β-cells when gastric inhibitory polypeptide (GIP) binds to and activates GIP receptor proteins on the pancreatic β-cells.

Although GIP also induces insulin secretion, there are similarly several drawbacks and disadvantages associated with solely activating the GIP receptor proteins on the pancreatic β-cells.

A major disadvantage associated with solely activating the GIP receptor proteins on the pancreatic β-cells is that GIP also induces glucagon secretion from pancreatic β-cells. Since glucagon is a hyperglycemic compound that increases blood sugar when secreted, the increase in glucagon secretion induced by GIP limits its therapeutic potential for treating abnormal insulin secretion.

To address the above noted disadvantage associated with solely activating the GIP receptor proteins on the pancreatic β-cells, Eli Lilly has recently developed a dual GLP-1/GIP analog that activates GLP-1 and GIP receptors on pancreatic β-cells.

The dual GLP-1/GIP analog, i.e., tirzepatide, which is marketed under the tradenames Mounjaro® and ZepBound®, provides the beneficial metabolic activity induced by both GLP-1 and GIP in a synergistic manner without a clinically significant increase in glucagon secretion.

Many of the drawbacks and disadvantages associated with administration of the GLP-1 analogs to patients are, however, also associated with administration of the dual GLP-1/GIP analog to patients.

Such drawbacks and disadvantages include the significant side effects that are often presented by the patients, including nausea, vomiting, diarrhea, abdominal pain, kidney problems and constipation, and high cost.

There is thus a need for compounds and ligands; particularly, natural compounds and ligands, and compositions comprising same, for treating physiological disorders; particularly, insulin-resistance-induced physiological disorders, such as type 2 diabetes mellitus, and cardiovascular disorders, which substantially reduce or overcome the drawbacks and disadvantages associated with administration (or delivery) of GLP-1 analogs and dual GLP-1/GIP analogs to a patient presenting with a physiological disorder.

It is thus one object of the present invention to provide natural compounds and ligands, and compositions comprising same, for treating physiological disorders; particularly, insulin-resistance-induced physiological disorders, such as type 2 diabetes mellitus, and cardiovascular disorders, which substantially reduce or overcome the drawbacks and disadvantages associated with administration (or delivery) of GLP-1 analogs and dual GLP-1/GIP analogs to a patient presenting with a physiological disorder.

It is another object of the present invention to provide natural compounds and ligands, and compositions comprising same, which, when administered to a patient presenting with a physiological disorder, effectively treat the physiological disorder by modulating ectopic olfactory receptor activity.

It is another object of the present invention to provide natural compounds and ligands, and compositions comprising same, which effectuate olfactory receptor (OR)-mediated secretion of endogenous GLP-1 and PYY in a patient presenting with a physiological disorder, without the undesirable side effects associated with delivery of a GLP-1 analog and/or a dual GLP-1/GIP analog to patients.

It is another object of the present invention to provide natural compounds and ligands, and compositions comprising same, which effectuate OR-mediated, free fatty acid receptor-mediated, and transient potential ion channel-mediated secretion of endogenous GLP-1, PYY and GIP in a subject presenting with a physiological disorder, without the undesirable side effects associated with delivery of a GLP-1 analog and/or a dual GLP-1/GIP analog to patients.

It is another object of the present invention to provide natural compounds and ligands, and compositions comprising same, which, when administered to a patient, effectively and safely modulate the patient's systemic insulin secretion in vivo, and can be administered to the patient via oral, sublingual, inhalation, intranasal, epidural, intracerebral, transdermal, topical, and injection administration means.

It is another object of the present invention to provide natural compounds and ligands, and compositions comprising same, which, when administered to a patient presenting with an insulin-resistance-induced physiological disorder; particularly, type 2 diabetes mellitus, effectively and safely modulate the patient's insulin secretion in vivo, whereby the insulin-resistance-induced physiological disorder is effectively treated and at least one risk factor associated with the insulin-resistance-induced physiological disorder is ameliorated with minimal, if any, adverse side effects.

It is another object of the present invention to provide natural compounds and ligands, and compositions comprising same, which, when administered to a patient presenting with a cardiovascular disorder, effectively and safely modulate the patient's insulin secretion in vivo, whereby the cardiovascular disorder is effectively treated and at least one risk factor associated with the cardiovascular disorder is ameliorated with minimal, if any, adverse side effects.

It is another object of the present invention to provide natural compounds and ligands, and compositions comprising same, which, when administered to a patient presenting with a cardiovascular disorder, effectively and safely modulate the patient's insulin secretion and abates 5-HT₂-serotonergic receptor activity in vivo, whereby the cardiovascular disorder is effectively treated and at least one risk factor associated with the cardiovascular disorder is ameliorated with minimal, if any, adverse side effects.

It is another object of the present invention to provide natural compounds and ligands, and compositions comprising same, which, when administered to a patient presenting with a cardiovascular disorder and an insulin-resistance-induced physiological disorder; particularly, type 2 diabetes mellitus, effectively and safely modulate the patient's insulin secretion in vivo, whereby the cardiovascular disorder and/or insulin-resistance-induced physiological disorder is effectively treated and at least one risk factor associated with the cardiovascular disorder or insulin-resistance-induced physiological disorder is ameliorated with minimal, if any, adverse side effects.

### SUMMARY OF THE INVENTION

The present invention is directed to compositions and methods for treating insulin-resistance-induced physiological disorders; particularly, cardiovascular diseases and disorders, and ameliorating physiological risk factors and seminal pathophysiological effects associated therewith.

In some embodiments of the invention, there are thus provided compositions for treating atherosclerosis presented by a patient.

In some embodiments, a composition for treating atherosclerosis presented by a patient comprises:
(i) at least a first receptor activating compound adapted to bind to and activate at least a first receptor selected from the group comprising olfactory receptor family 51 subfamily E member 1 (OR51E1) and olfactory receptor family 1 subfamily A member 1 (OR1A1), and
(ii) at least a second receptor activating compound adapted to bind to and activate at least a second receptor selected from the group comprising free fatty acid receptor 1 (FFAR1) and free fatty acid receptor 4 (FFAR4), the composition adapted to modulate insulin secretion in vivo, whereby the atherosclerosis is treated.

In a preferred embodiment, the composition is further adapted to ameliorate at least one risk factor associated with the atherosclerosis when the composition is delivered to the patient.

In some embodiments, the composition is further adapted to ameliorate at least one seminal pathophysiological effect associated with the atherosclerosis when the composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the composition.

In some embodiments, the first compound comprises 3-methylpentanoic acid.

In some embodiments, the 3-methylpentanoic acid comprises an EC₅₀ value of at least approximately 0.1 μM in the composition.

In a preferred embodiment, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the composition.

In some embodiments, the composition further comprises at least a third receptor activating compound adapted to bind to and activate at least a third receptor selected from the group comprising olfactory receptor family 2 subfamily B member 11 (OR2B11), olfactory receptor family 2 subfamily J member 3 (OR2J3) and transient receptor potential cation channel subfamily A member 1 (TRPA1).

In some embodiments, the third receptor activating compound comprises cinnamaldehyde.

In some embodiments, the cinnamaldehyde comprises an EC₅₀ value of at least approximately 0.1 μM in the composition.

In some embodiments, the composition further comprises at least a fourth receptor activating compound adapted to bind to and restrict 5-HT₂-serotongeric receptor activity.

In some embodiments, the fourth receptor activating compound comprises a fourth compound selected from the group comprising 3,3′,4′,5,6,7,8-heptamethoxyflavone (HMF), hesperidin and isoliquiritigenin (ISL).

In some embodiments, the fourth compound comprises HMF.

In some embodiments, the HMF comprises an EC₅₀ value of at least approximately 0.01 μM in the composition.

In a preferred embodiment, the first receptor activating compound is adapted to induce at least 50% activation of at least olfactory receptor olfactory receptor OR51E1 in vivo when the composition is delivered to the patient.

In some embodiments, a composition for treating atherosclerosis presented by a patient comprises:
(i) at least a first receptor activating compound adapted to bind to and activate at least a first receptor selected from the group comprising olfactory receptor family 51 subfamily E member 1 (OR51E1) and olfactory receptor family 1 subfamily A member 1 (OR1A1),
(ii) at least a second receptor activating compound adapted to bind to and activate at least a second receptor selected from the group comprising free fatty acid receptor 1 (FFAR1) and free fatty acid receptor 4 (FFAR4), and
(iii) at least a second receptor activating compound adapted to bind to and activate olfactory receptor OR2L13,
the composition adapted to modulate insulin secretion in vivo, whereby the atherosclerosis is treated.

In a preferred embodiment, the composition is further adapted to ameliorate at least one risk factor associated with the atherosclerosis when the composition is delivered to the patient.

In some embodiments, the composition is further adapted to ameliorate at least one seminal pathophysiological effect associated with the atherosclerosis when the composition is delivered to the patient.

In some embodiments, the first receptor activating compound comprises a first compound selected from the group comprising eugenol and geraniol.

In some embodiments, the first compound comprises eugenol.

In some embodiments, the eugenol comprises an EC₅₀ value of at least approximately 0.1 μM in the composition.

In some embodiments, the first compound comprises geraniol.

In some embodiments, the geraniol comprises an EC₅₀ value of at least approximately 0.1 μM in the composition.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the composition.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In some embodiments, the third receptor activating compound comprises a fourth compound selected from the group comprising (−)-carvone and choline.

In some embodiments, the fourth compound comprises (−)-carvone.

In some embodiments, the (−)-carvone comprises an EC₅₀ value of at least approximately 0.1 μM in the GLP-1/GIP secretion composition.

In some embodiments, the composition further comprises at least a fourth receptor activating compound adapted to bind to and restrict 5-HT₂-serotongeric receptor activity.

In some embodiments, the fourth receptor activating compound comprises a fifth compound selected from the group comprising 3,3′,4′,5,6,7,8-heptamethoxyflavone (HMF), hesperidin and isoliquiritigenin (ISL).

In some embodiments, the fifth compound comprises HMF.

In some embodiments, the HMF comprises an EC₅₀ value of at least approximately 0.01 μM in the composition.

In a preferred embodiment, at least one of the first receptor activating compounds is adapted to induce at least 50% activation of at least olfactory receptor olfactory receptor OR51E1 in vivo when the composition is delivered to the patient.

In a preferred embodiment, at least one of the third receptor activating compounds is adapted to induce at least 50% activation of olfactory receptor OR2L13 in vivo when the composition is delivered to the patient.

In some embodiments, wherein the patient also presents with an insulin-resistance-induced physiological disorder; particularly, type 2 diabetes mellitus, the compositions referenced above are also adapted to treat the atherosclerosis and insulin-resistance-induced physiological disorder, when the composition is delivered to the patient.

In some embodiments of the invention, there are thus also provided methods for treating atherosclerosis presented by a patient.

In some embodiments, a method for treating atherosclerosis presented by a patient comprises the steps of:
(a) providing one of the compositions referenced above; and
(b) delivering the composition to the patient, wherein the patient's insulin secretion is modulated in vivo, whereby the atherosclerosis is effectively treated.

In some embodiments, a method for treating atherosclerosis presented by a patient comprises the steps of:
(a) providing one of the compositions referenced above; and
(b) delivering the composition to the patient, wherein the patient's insulin secretion is modulated and 5-HT₂-serotongeric receptor activity is restricted in vivo, whereby the atherosclerosis is effectively treated.

In some embodiments of the invention, there are also provided methods for treating atherosclerosis and an insulin-resistance-induced physiological disorder presented by a patient.

In some embodiments, a method for treating atherosclerosis and an insulin-resistance-induced physiological disorder presented by a patient comprises the steps of:
(a) providing one of the compositions referenced above; and
(b) delivering the composition to the patient, wherein the patient's insulin secretion is modulated in vivo, whereby the atherosclerosis and insulin-resistance-induced physiological disorder are effectively treated.

In some embodiments of the invention, the insulin-resistance-induced physiological disorder treated by the compositions comprises type 2 diabetes mellitus.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of receptor-mediated activation of GLP-1 secretion from endogenous gastrointestinal cells; and

FIG. 2 is a schematic illustration of receptor-mediated activation of GIP secretion from endogenous gastrointestinal cells.

### DETAILED DESCRIPTION OF THE PREFERRED EMBODIMENT

Before describing the present invention in detail, it is to be understood that this invention is not limited to particularly exemplified compounds, compositions or methods, as such may, of course, vary. Thus, although a number of compounds, compositions and methods similar or equivalent to those described herein can be used in the practice of the present invention, the preferred compounds, compositions and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a”, “an” and “the” include plural referents unless the content clearly dictates otherwise. Thus, for example, reference to “an active agent” includes two or more such agents and the like.

### Definitions

The term “olfactory receptor (OR)” as used herein, means and includes an olfactory receptor that is a seminal component of the chemosensory organs responsible for olfaction. The term “olfactory receptor” as used herein, also means, and includes, trace amine associated receptors, vomeronasal receptors, formyl peptide receptors, membrane guanylyl cyclase, subtype GC-D receptors; and G-protein coupled receptors, such as G-protein coupled taste receptors. Olfactory receptors can also include hybrid receptors synthesized from the above-noted olfactory receptors.

The term “ectopic olfactory receptor”, as used herein, means and includes an olfactory receptor that is present in organs, tissue, and/or cells that is a seminal component of physiological processes outside of olfaction and, in some instances, indirectly involved with olfactory-mediated processes.

The terms “olfaction” and “olfactory reception” are used interchangeably herein, mean and include the interaction of a composition (or formulation) with an olfactory receptor coupled to a cell signaling pathway. The composition can also be defined as an “odorant” and may be airborne (i.e., volatile) and/or in solution.

The term “free fatty acid receptor”, as used herein, means and includes a transmembrane cell surface receptor that is adapted and configured to bind to fatty acids and induce cell signaling processes in response to the binding of the fatty acids.

The term “transient receptor potential ion channel”, as used herein, means and includes a transmembrane ion channel that is adapted and configured to modulate ion entry into an endogenous cell, such as Ca²⁺ entry, and, thereby, induce cell signaling process when a compound or ligand binds to the ion channel.

The term “insulin resistance”, as used herein, means, and includes a condition in which insulin exerts a biological effect that is lower than expected, due to defects in insulin-stimulated glucose uptake; particularly, in glycogen synthesis and, to a lesser extent, glucose oxidation.

The term “insulin-resistance-induced physiological disorder”, as used herein, thus means and includes a physiological disease and/or a physiological disorder characterized by metabolic dysfunction and conditions associated therewith including, but not limited to, dysfunction of glucose metabolism and attendant insulin resistance.

The term “modulation,” as used herein in connection with insulin, means and includes, without limitation, activating and/or regulating at least one cellular process relating to production, synthesis and transmission of insulin, including, but not limited to (i) production and, hence, release of the insulin via activation of a receptor, e.g., GLP-1 receptor and GIP receptor activation, (ii) binding of the released insulin to insulin receptors, (iii) synthesis of the insulin, (iv) alteration(s) of intracellular signaling pathways associated with insulin synthesis and secretion and (v) alteration(s) of cellular sensitivity to extracellular insulin.

The term “agonist” as used herein, means, and includes any molecule which binds to a receptor on a cell, wherein the binding to the receptor can potentially lead to subsequent changes in the cell's functions. When an agonist binds to a sufficient number of receptors, the receptors can activate seminal processes in the cell.

The term “antagonist”, as used herein, means and includes a molecule and/or a compound comprising same, which binds to a receptor on a cell and inhibits the receptor from activating processes in the cell. The inhibition of the receptor can include competitive binding against agonists (when an antagonist is bound, agonists cannot bind to the receptor) and allosteric effects (when the antagonist binds, agonists can still bind the receptor, but cannot activate the receptor).

The term “endocrine factor” as used herein, means and includes any molecule and/or a compound comprising same, which is produced and secreted by endogenous cells and induces biological activity at a biological tissue site. The term “endocrine factor” thus means and includes, without limitation, insulin, glucagon-like peptide-1 (GLP-1), gastric inhibitory polypeptide (GIP), peptide Y-Y (PYY), ghrelin, gastrin, cholecystokinin (CCK), bombesin/gastrin releasing peptide (BBS/GRP), neurotensin (NT), glucagon-like peptide 2 (GLP-2), calcitonin gene-related peptide (CGRP), chromogranin A, enteroglucagon, galanin, leptin, motilin, amylin, neuropeptide Y (NPY), pancreatic polypeptide, substance P, oxyntomodulin, and somatostatin.

The term “dysregulation,” as used herein in connection with an endocrine factor, means and includes abnormality or impairment in the synthesis, production and/or transmission of an endocrine factor and, hence, abnormality or impairment in biological processes modulated by the endocrine factor.

The term “compound”, as used herein, means and includes any composition of matter comprising two or more chemical elements. According to the invention, in some instances, the terms “compound” and “ligand” are synonymous and used interchangeably herein.

The term “compound” thus means and includes, without limitation, the following natural compounds and ligands (referred to herein as “receptor activating compounds and ligands): pentanoic acid, pentanol, 3-methylnonanoic acid, 4-methylnonanoic acid, 3-methyl-2,4 nonanedione, eugenol, farnesol, farnesyl thiosalicylic acid, acrolein, formalin, hydrogen peroxide, coumarin, dicyclohexyl disulfide, nonanoic acid, octanoic acid, 2-nonanoic acid, butyric acid, 2-heptanone, heptanoic acid, decanoic acid, tetradecanoic acid, trans-2-decenoic acid, tridecanoic acid, undecanoic acid, methyl eugenol, methyl salicylate, (+)-menthol, eugenyl acetate, 2,4-dinitrotoluene, 4-hydroxynonenal, hexanoic acid, 2-ethylhexanoic acid, 2-ethyl-3,5-dimethylpyrazine, pyrazine, dimethyl disulfide, methyl furfuryl disulfide, propanal, butyl butyryl lactate, isovaleric acid, propionic acid, 4-methylpentanoic acid, methanoic acid, coumarin, acrolein, helional, lilial, β-ionone, androstenone, androstadienone, caramel furanone, 3-phenyl propyl propionate, ethyl vanillin, 2-ethyl-fencol, N-amyl acetate, eugenol acetate, sandalwood, S-(−)-citronellol, (−)-citronellol, hydroxycitronellal, citral, S-(−)-citronellal, geraniol, estragole, neroli, heptanol, octanol, nonanal, (+)-carvine, (−)-carvone, choline, 3,3′,4′,5,6,7,8-heptamethoxyflavone (HMF), hesperidin, isoliquiritigenin, linalool, bourgeonal, acetophenone, amyl butyrate, nonanethiol, allyl phenyl acetate, N-amyl acetate, muscone, isoeugenol, eugenol methyl ether, heptanol, hexanol, hexanal, hexyl acetate, 1-hexanol, 1-heptanol, 2-heptanone, octanal, octanol, 1-octanol, 1-octanal, musk ketone, (+)-dihydrocarvone, α-cedrene, celery ketone, anis aldehyde, vanillin, guaiacol, thujopsene, hydroxymethylpentylcyclohexene-carboxaldehyde (lyral), allyl phenylacetate, allyl isothiocyanate, benzyl acetate, 3,4-hexanedione, cis-3-hexen-1-ol, quinoline, ethyl heptanoate, methyl octanoate, nonanal, 1-nonanol, 2-nonanol, 3-octanone, 3-nonanone, decyl aldehyde, (E)-non-2-enal, 2-ethyl-3,5-dimethylpyrazine, 3-methylbut-2-ene-1-thiol, (2E,6Z)-nona-2,6-dienalcitral, ethyl octanoate, octanoate, p-mentha-8-thiol-3-one, β-myrcene, γ-decalactone, (S)-(+)-carvone, dihydrojasmone, dicyclohexyl disulfide, cinnamaldehyde, spearmint, coffee difuran, quinoline, butyl anthranilate 2,2-dithiodimethyl-enedifuran, ethyl hexanoate, limonene, α-terpineol, eugenol (3E,5Z)-undeca-1,3,5-triene, acetate, butyrate, nicotinic acid, long-chain free fatty acids (e.g., palmitic acid and stearic acid), medium-chain free fatty acids (e.g., caproic acid (C6:0), caprylic acid (C8:0), capric acid (C10:0), and lauric acid (C12:0)), and omega-3 polyunsaturated fatty acids (e.g., alpha-linoleic acid, docosahexaenoic acid and eicosatetraenoic acid).

The term “compound” also means and includes any composition of matter included in the Food and Drug Administration's (FDA's) generally recognized as safe (GRAS) database.

The term “natural,” as used herein in connection with a compound or ligand, and a composition of the invention formed therewith, means and includes a compound or ligand that exists or is synthesized in nature without intervention, including, by way of example, a food molecule.

The term “natural,” as used herein in connection with a compound or ligand, and a composition of the invention formed therewith, also means and includes a compound or ligand that originally existed or was synthesized in nature without intervention and is subjected to processing without altering the chemical structure of the compound, such as chemical purification and isolation processes and the formulation of compositions from two or more compounds.

The terms “composition”, “formulation”, “olfactory composition” and “olfactory formulation” are used interchangeably herein, mean and include any compound or combination of compounds that can interact with and modulate at least one olfactory receptor and/or ectopic olfactory receptor and/or free fatty acid receptor and/or transient receptor potential ion channel.

The terms “express” and “expression” as used interchangeably herein, mean, and include the production of a protein product from the genetic information contained within a nucleic acid sequence.

The term “upregulation”, as used herein, means, and includes the increased production of a protein product from the genetic information contained within a nucleic acid sequence.

The term “downregulation”, as used herein, means, and includes the decreased production of a protein product from the genetic information contained within a nucleic acid sequence.

The terms “prevent” and “preventing” are used interchangeably herein, and mean and include precluding a disease, physiological disorder, or pathological condition presented by a subject or patient. The term does not require an absolute preclusion of the disease or condition. Rather, this term includes decreasing the chance for disease occurrence and recurrence.

The terms “prevent” and “preventing” also mean and include reducing the frequency or severity of a disease, physiological disorder or pathological condition presented by a subject or patient.

The terms “treat,” “treatment” and “treating” are used interchangeably herein, and mean and include management of a disease, physiological disorder or pathological condition presented by a subject or patient to cure, ameliorate, stabilize, or prevent the disease, physiological disorder or pathological condition. The terms “treat” and “treating” include “active treatment”, i.e., treatment intended to cure or stabilize a disease, physiological disorder or pathological condition, and “causal treatment”, i.e., treatment intended to abate or prevent the cause of the associated disease, physiological disorder or pathological condition.

The terms “treat,” “treatment” and “treating” further include “palliative treatment”, i.e., treatment intended to relieve the symptoms a disease, physiological disorder or pathological condition, “preventative treatment”, i.e., treatment intended to minimize or partially or completely inhibit the development of the associated disease, physiological disorder or pathological condition, and “supportive treatment”, i.e., treatment employed to supplement another treatment modality directed toward curing, ameliorating, stabilizing, or preventing the associated disease, pathological condition, or disorder.

The terms “delivery” and “administration” are used interchangeably herein, and mean and include providing a composition (or formulation), through any method appropriate to deliver the composition (or formulation) to a subject. According to the invention, such administration means includes, without limitation, oral, sublingual, inhalation, intranasal, epidural, intracerebral, transdermal, topical, and injection administration means.

The term “IC₅₀”, as used herein, means, and includes the concentration of an agonist, antagonist, compound and/or ligand, which, after delivery, inhibits or attenuates at least 50% of a biological and/or physiological process.

In some embodiments, the term “IC₅₀” refers to the concentration of a modulator (e.g., an antagonist or compound), which, after delivery, abates, inhibits or attenuates at least 50% of a biological and/or physiological process, e.g., lipogenesis, atherosclerotic plaque formation, maladaptive RAAS activation, and maladaptive remodeling of vascular or myocardial tissue.

The term “EC₅₀”, as used herein, means, and includes the concentration of an agonist, compound and/or ligand, which, after delivery to a subject, induces at least 50% activation of a biological and/or physiological process.

In some embodiments, the term “adapted”, as used in connection with a “compound”, “ligand”, “agonist”, and “antagonist”, means the “compound”, “ligand”, “agonist” and/or “antagonist” is capable of inducing or attenuating one or more biological and/or physiological processes or activities, including, without limitation, (i) activating or antagonizing a receptor, including, without limitation, adipose olfactory receptors, central nervous system (CNS) olfactory receptors, cardiovascular olfactory receptors, trace amine-associated receptors, gastrointestinal (GI) olfactory receptors, free fatty acid receptors, transient receptor potential ion channels, dopaminergic neuron olfactory receptors and serotonergic receptors and/or (ii) inducing or attenuating synthesis, secretion and transmission of a molecule or macromolecule, including, without limitation, β-secretase (BACE1), glucagon-like peptide-1 (GLP-1), peptide Y-Y (PYY), gastric inhibitory polypeptide (GIP), serotonin (5-HT), dopamine, secretin, prostaglandin E₂, vasoactive intestinal protein (VIP), nuclear factor κβ(NK-κβ) and an NADPH oxidase (NOX) by virtue of the concentration, i.e., IC₅₀ or EC₅₀ value, of the “compound”, “ligand”, “agonist”, or “antagonist.”

In some embodiments, the term “adapted”, as used in connection with a “composition” and “formulation” also means the “composition” and/or “formulation” is capable of inducing or attenuating one or more biological and/or physiological processes or activities, including, without limitation, (i) activating or antagonizing a receptor, including, without limitation, adipose olfactory receptors, central nervous system (CNS) olfactory receptors, cardiovascular olfactory receptors, trace amine-associated receptors, gastrointestinal (GI) olfactory receptors, free fatty acid receptors, transient receptor potential ion channels, dopaminergic neuron olfactory receptors and serotonergic receptors and/or (ii) inducing or attenuating synthesis, secretion and transmission of a molecule or macromolecule, including, without limitation, β-secretase (BACE1), glucagon-like peptide-1 (GLP-1), peptide Y-Y (PYY), gastric inhibitory polypeptide (GIP), serotonin (5-HT), dopamine, secretin, prostaglandin E₂, vasoactive intestinal protein (VIP), nuclear factor κβ (NK-κβ) and an NADPH oxidase (NOX) by virtue the concentration, i.e., IC₅₀ or EC₅₀ value, of a “compound” or “ligand” contained in the “composition” or “formulation.”

In some embodiments, the term “adapted”, as used in connection with a “composition” and “formulation” also means the “composition” and/or “formulation” is capable of inducing or abating one or more biological and/or physiological processes or activities referenced above by virtue of the concentrations, i.e., IC₅₀ or EC₅₀ values, of two (2) or more “compounds”, “ligands”, “agonists”, or “antagonists” in the “composition” or “formulation.”

The term “comprise” and variations of the term, such as “comprising” and “comprises”, means “including, but not limited to” and is not intended to exclude, for example, other compounds, ligands or method steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention.

The present invention is directed to compositions and methods for treating cardiovascular disorders and insulin-resistance-induced physiological disorders; particularly, type 2 diabetes mellitus, by modulating receptor activity and, thereby, insulin secretion in vivo.

As discussed above, various entities have developed GLP-1 analogs that mimic endogenous GLP-1 alone, and dual GLP-1/GIP analogs that mimic both endogenous GLP-1 and GIP in combination, which, when delivered to a patient, induce secretion of endogenous GLP-1 and GIP, and, thereby, insulin secretion.

As also discussed above, the GLP-1 analogs activate the GLP-1 receptor on pancreatic β-cells and the dual GLP-1/GIP analogs activate both GLP-1 receptor and GIP receptor on pancreatic β-cells to modulate insulin secretion by the pancreas.

Although the GLP-1 analogs and dual GLP-1/GIP analogs can effectively modulate insulin secretion, as also discussed in detail above, there are several drawbacks and disadvantages associated with administration of GLP-1 analogs and dual GLP-1/GIP analogs to patients, including, a high risk of hypoglycemia, adverse side effects, and high costs.

As discussed in detail below, Applicant has developed compositions, which, when delivered to a patient, effectively and safely modulate endogenous GLP-1 and GIP secretion and, thereby, insulin secretion in vivo, which (i) overcome the drawbacks and disadvantages associated with delivery of GLP-1 analogs, which merely mimic endogenous GLP-1, and dual GLP-1/GIP analogs, which merely mimic endogenous GLP-1 and GIP, and (ii) can be readily employed to treat a multitude of physiological disorders.

Although the compositions of the invention are described in connection with the treatment of cardiovascular diseases and disorders and underlying causes thereof, use of the compositions is not limited solely to the treatment of cardiovascular diseases and disorders, and underlying causes thereof. As set forth in Applicant's U.S. application Ser. Nos. 18/430,796 and 18/892,760, which are expressly incorporated by reference herein in their entirety, the compositions can also be employed to effectively treat various insulin-resistance-induced physiological disorders, such as type 2 diabetes mellitus.

As will readily appreciated by one having ordinary skill in the art, the compositions can also be employed to effectively treat a multitude of other physiological diseases and disorders, including, without limitation, liver diseases and disorders, e.g., fatty liver disease, psychopathological diseases and disorders, reproductive diseases and disorders, and immune diseases and disorders.

As discussed in detail herein, in preferred embodiment, the compositions of the invention comprise at least one natural compound or ligand that is adapted to bind to and activate (and, hence, modulate) at least one receptor, e.g., an ectopic olfactory receptor and/or free fatty acid receptor and/or transient receptor potential ion channel, whereby GLP-1 and/or peptide Y-Y (PYY) and/or GIP secretion is induced and, thereby, insulin secretion is modulated in vivo.

### Receptor Activating Compounds and Ligands

According to the invention, suitable natural compounds and ligands, which are adapted to modulate insulin secretion via induced receptor activity, include, without limitation, the natural receptor activating compounds and ligands referenced above.

As discussed in detail herein, in some embodiments of the invention, the preferred natural receptor activating compounds and ligands of the invention that are adapted to modulate insulin secretion via induced receptor activity, include, without limitation, pentanol, 3-methylnonanoic acid, 4-methylnonanoic acid, 3-methyl-2,4 nonanedione, eugenol, farnesol, (β-ionone, isovaleric acid, propionic acid, farnesyl thiosalicylic acid, acrolein, formalin, hydrogen peroxide, coumarin, nonanoic acid, octanoic acid, 2-nonanoic acid, 2-heptanone, 4-hydroxynonenal, butyl butyryl lactate, isovaleric acid, coumarin, acetate, butyrate, nicotinic acid, eugenol acetate, S-(−)-citronellol, (−)-citronellol, citral, S-(−)-citronellal, geraniol, estragole, neroli, heptanol, octanol, nonanal, hexanol, hexanal, hexyl acetate, 1-hexanol, 1-heptanol, octanal, octanol, 1-octanol, 1-octanal, musk ketone, (+)-dihydrocarvone, α-cedrene, thujopsene, lyral, allyl phenylacetate, allyl isothiocyanate, benzyl acetate, 3,4-hexanedione, cis-3-hexen-1-ol, quinoline, 3-octanone, 2-ethyl-3,5-dimethylpyrazine, dicyclohexly disulfide, cinnamaldehyde, spearmint, coffee difuran, palmitic acid, stearic acid, caproic acid (C6:0), caprylic acid (C8:0), capric acid (C10:0), lauric acid (C12:0), alpha-linoleic acid, docosahexaenoic acid and eicosatetraenoic acid.

In a preferred embodiment, the natural receptor activating compounds and ligands of the invention comprise a molecular weight less than approximately 500.0 Da, whereby the natural receptor activating compounds and ligands can cross the blood-brain-barrier.

According to the invention, the natural receptor activating compounds and ligands of the invention (and, hence, compositions of the invention formed therefrom) are adapted to bind to and activate one or more of the following receptors: adipose olfactory receptors, adrenal olfactory receptors, central nervous system (CNS) olfactory receptors, dopaminergic neuron olfactory receptors, mammary olfactory receptors, cardiovascular olfactory receptors, renal olfactory receptors, hepatic olfactory receptors, lymphatic olfactory receptors, ovarian olfactory receptors, prostate olfactory receptors, dermal olfactory receptors, testicular olfactory receptors, hematologic olfactory receptors, trace amine-associated receptors, gastrointestinal (GI) olfactory receptors, free fatty acid receptors, and transient receptor potential ion channels.

In some embodiments of the invention, the natural receptor activating compounds and ligands of the invention referenced above and, hence, compositions of the invention formed therefrom are adapted to bind to and activate combinations of the aforementioned receptors, i.e., multiple receptors.

As discussed in detail herein, in a preferred embodiment, the natural receptor activating compounds and ligands of the invention (and, hence, compositions of the invention formed therefrom) are adapted to bind to and activate at least one of the following receptors: olfactory receptor family 51 subfamily E member 1 (OR51E1), olfactory receptor family 1 subfamily A member 1 (OR1A1), olfactory receptor family 2 subfamily C member 1 (OR2C1), olfactory receptor family 10 subfamily J member 5 (OR10J5), free fatty acid receptor 1 (FFAR1), free fatty acid receptor 4 (FFAR4), olfactory receptor family 2 subfamily W member 1 (OR2W1), olfactory receptor family 2 subfamily B member 11 (OR2B11), olfactory receptor family 2 subfamily J member 3 (OR2J3), and transient receptor potential cation channel subfamily A member 1 (TRPA1).

As set forth in Applicant's priority U.S. application Ser. No. 18/430,796 and discussed further below, when one of the natural receptor activating compounds and ligands referenced above (and composition formed therewith) binds to and activates olfactory receptor OR51E1, olfactory receptor OR1A1, olfactory receptor OR2C1, olfactory receptor OR10J5, olfactory receptor OR2W1, olfactory receptor OR2B11, olfactory receptor OR2J3, free fatty acid receptor FFAR1, free fatty acid receptor 4 (FFAR4) and/or transient receptor TRPA1, GLP-1 and/or peptide Y-Y (PYY) and/or GIP secretion is induced and, thereby, insulin secretion is modulated in vivo.

### Olfactory Receptor-Mediated GLP-1 and PYY Secretion

As indicated above, when one of the natural receptor activating compounds and ligands of the invention (and composition formed therewith) binds to and activates at least olfactory receptor OR51E1, olfactory receptor OR1A1, olfactory receptor OR2C1 or olfactory receptor OR10J5, GLP-1 and/or PYY secretion is induced in vivo.

The compositions formed from the noted natural receptor activating compounds and ligands are thus referred to herein as “GLP-1/PYY secretion compositions” (and, in some instances “GLP-1/GIP secretion compositions”).

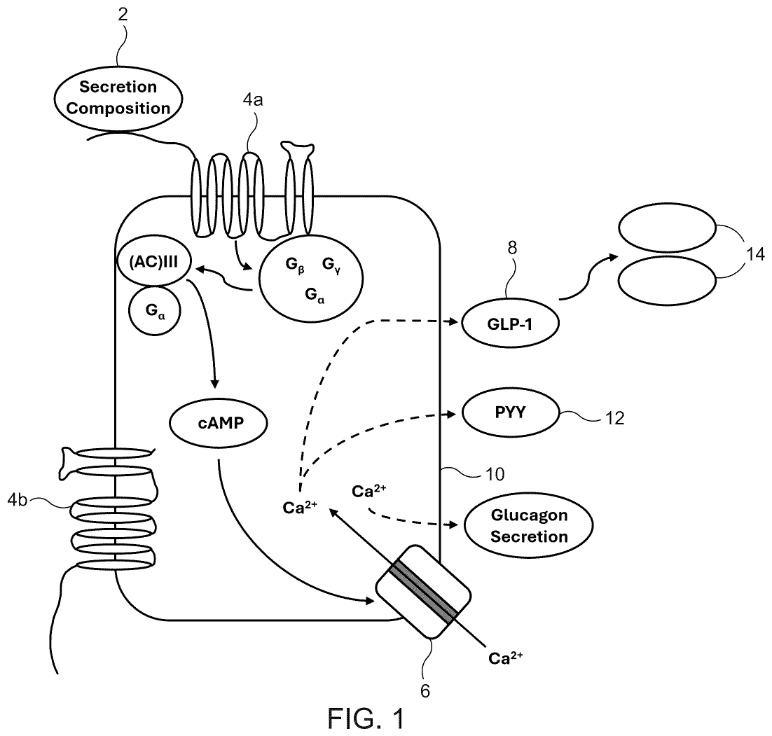

Referring to FIG. 1, according to the invention, when a GLP-1/PYY secretion composition (and, in some instances, a GLP-1/GIP secretion composition) of the invention (denoted “2”) is delivered to a subject or patient, the GLP-1/PYY secretion composition 2 binds to at least one of the ectopic olfactory receptors (ORs) referenced above (denoted “4a, 4b”) disposed on enteroendocrine cells and pancreatic α-cells, in this instance, enteroendocrine L-cells, as depicted in FIG. 1, wherein a representative enteroendocrine L-cell is denoted “10”.

As illustrated in FIG. 1, the binding of the GLP-1/PYY secretion composition 2 to at least one of the ORs 4a, 4b activates the ORs 4a, 4b, in this instance 4a, by inducing a conformational change in the molecular structure of OR 4a, which activates intracellular G_{α}/G_{β}/G_{γ} subunits of OR 4a, whereby the G_{α}/G_{β}/G_{γ} subunits act as a guanine nucleotide exchange factor, wherein a guanine diphosphate (GDP) is exchanged for a guanine triphosphate (GTP), which binds to the Ga subunit of OR 4a.

As further illustrated in FIG. 1, the noted binding of the GTP to the Ga subunit induces a dissociation of the G_{α}/G_{β}/G_{γ} subunits of OR 4a into a (i) free G_{α} subunit, which binds to adenyl cyclase (AC) III, and a (ii) G_{β}/G_{γ} complex and, thereby, activates seminal downstream cell signaling processes that induce an increase in intracellular cAMP in the enteroendocrine L-cell 10 and, hence, cells.

The noted increase in intracellular cAMP and a glucose-induced membrane depolarization of the enteroendocrine L-cell 10 (and, hence, cells) opens the voltage-dependent Ca²⁺ (VDC) channels (denoted “6”) of the enteroendocrine L-cells 10, and the resulting Ca²⁺ influx triggers vesicular exocytosis and increases secretion of GLP-1 (denoted “8”) from the enteroendocrine L-cells 10 (and, in some instances, α-cells). In some instances, e.g., when at least one of the activated ORs comprise OR51E1, the resulting Ca²⁺ influx triggers vesicular exocytosis and also increases secretion of PYY (denoted “12”) from the enteroendocrine L-cells 10.

As further illustrated in FIG. 1, the secreted GLP-1 8 binds to and activates GLP-1 receptor proteins on pancreatic β-cells (denoted “14”), which, as indicated above, induces secretion of insulin therefrom.

As indicated above, the GLP-1/PYY secretion compositions of the invention comprise at least one receptor activating compound or ligand that is specifically adapted to bind to and activate at least olfactory receptor OR51E1, olfactory receptor OR1A1, olfactory receptor OR2C1 or olfactory receptor OR10J5, whereby, as indicated above, GLP-1 and/or PYY secretion is induced in vivo.

As discussed below, in some embodiments, the preferred natural receptor activating compounds (and/or ligands) of the GLP-1/PYY secretion compositions comprise 3-methylpentanoic acid, 4-methylpentanoic acid, farnesol, eugenol, nonanoic acid, pentanol, butyl butyryl lactate, isovaleric acid, geraniol, citronellol, 3-methyl-2,4-nonanedione, estragole, neroli, heptanol, octanol, helional, nonanal, hydroxycitronellal, citral, octanoic acid, eugenol, musk ketone, (+)-dihydrocarvone, α-cedrene, lyral and thujopsene.

According to the invention, the EC₅₀ values of the noted natural receptor activating compounds and ligands contained in a GLP-1/PYY secretion composition of the invention (and GLP-1/GIP secretion compositions of the invention, discussed below) can comprise any EC₅₀ values or EC₅₀ value ranges in the range of approximately 1.0 nM to approximately 200.0 mM.

Thus, according to the invention, the EC₅₀ values of natural receptor activating compounds and ligands contained in the GLP-1/PYY secretion compositions of the invention (and GLP-1/GIP secretion compositions of the invention) can comprise in the range of approximately 0.001 μM to approximately 100000.0 μM, approximately 0.002 μM to approximately 10000.0 μM, approximately 0.003 μM to approximately 1000.0 μM, approximately 0.005 μM to approximately 750.0 μM, approximately 0.01 μM to approximately 500.0 μM, approximately 0.05 μM to approximately 50.0 μM, approximately 0.05 μM to approximately 100.0 μM, approximately 0.05 μM to approximately 150.0 μM, approximately 0.05 μM to approximately 200.0 μM, approximately 0.05 μM to approximately 250.0 μM, approximately 0.05 μM to approximately 300.0 μM, approximately 0.05 μM to approximately 350.0 μM, approximately 0.05 μM to approximately 400.0 μM, approximately 0.05 μM to approximately 450.0 μM, approximately 0.05 μM to approximately 500.0 μM, approximately 0.1 μM to approximately 50.0 μM, approximately 0.1 μM to approximately 100.0 μM, approximately 0.1 μM to approximately 150.0 μM, approximately 0.1 μM to approximately 200.0 μM, approximately 0.1 μM to approximately 250.0 μM, approximately 0.1 μM to approximately 300.0 μM, approximately 0.1 μM to approximately 350.0 μM, approximately 0.1 μM to approximately 400.0 μM, approximately 0.1 μM to approximately 450.0 μM, approximately 0.1 μM to approximately 500.0 μM, approximately 0.1 μM to approximately 1000.0 μM, approximately 0.1 μM to approximately 1500.0 μM, approximately 0.1 μM to approximately 2000.0 μM, approximately 0.1 μM to approximately 2500.0 μM, approximately 0.1 μM to approximately 3000.0 μM, approximately 0.25 μM to approximately 50.0 μM, approximately 0.25 μM to approximately 100.0 μM, approximately 0.25 μM to approximately 150.0 μM, approximately 0.25 μM to approximately 200.0 μM, approximately 0.25 μM to approximately 250.0 μM, approximately 0.25 μM to approximately 300.0 μM, approximately 0.25 μM to approximately 350.0 μM, approximately 0.25 μM to approximately 400.0 μM, approximately 0.25 μM to approximately 450.0 μM, approximately 0.25 μM to approximately 500.0 μM, approximately 0.5 μM to approximately 300.0 μM, approximately 1.0 μM to approximately 50.0 μM, approximately 1.0 μM to approximately 100.0 μM, approximately 1.0 μM to approximately 150.0 μM, approximately 1.0 μM to approximately 200.0 μM, approximately 1.0 μM to approximately 250.0 μM, approximately 1.0 μM to approximately 300.0 μM, approximately 1.0 μM to approximately 350.0 μM, approximately 1.0 μM to approximately 400.0 μM, approximately 1.0 μM to approximately 450.0 μM, approximately 1.0 μM to approximately 500.0 μM, approximately 2.5 μM to approximately 100.0 μM, approximately 5.0 μM to approximately 75.0 μM, approximately 7.5 μM to approximately 50.0 μM, approximately 10.0 μM to approximately 25.0 μM, and/or any ECso values therebetween.

According to the invention, the EC₅₀ values of natural receptor activating compounds and ligands contained in the GLP-1/PYY secretion compositions of the invention (and GLP-1/GIP secretion compositions of the invention) can also comprise in the range of approximately 0.001 μM to approximately 10.0 μM, approximately 0.005 μM to approximately 7.5 μM, approximately 0.01 μM to approximately 5.0 μM, approximately 0.03 μM to approximately 2.5 μM, approximately 0.05 μM to approximately 1.5 μM, approximately 0.03 μM to approximately 1.0 μM, approximately 0.1 μM to approximately 0.5 μM, and/or any EC₅₀ values therebetween.

In some embodiments, the EC₅₀ values of natural receptor activating compounds and ligands contained in the GLP-1/PYY secretion compositions of the invention (and GLP-1/GIP secretion compositions of the invention) comprise at least 0.001 μM, at least 0.002 μM, at least 0.003 μM, at least 0.004 μM, at least 0.005 μM, at least 0.006 μM, at least 0.007 μM, at least 0.008 μM, at least 0.009 μM, at least 0.01 μM, at least 0.02 μM, at least 0.03 μM, at least 0.04 μM, at least 0.05 μM, at least 0.06 μM, at least 0.07 μM, at least 0.08 μM, at least 0.09 μM, at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, at least 1.0 μM, at least 2.0 μM, at least 3.0 μM, at least 4.0 μM, at least 5.0 μM, at least 6.0 μM, at least 7.0 μM, at least 8.0 μM, at least 9.0 μM, at least 10.0 μM, at least 20.0 μM, at least 30.0 μM, at least 40.0 μM, at least 50.0 μM, at least 60.0 μM, at least 70.0 μM, at least 80.0 μM, at least 90.0 μM, at least 100.0 μM, at least 200.0 μM, at least 300.0 μM, at least 400.0 μM, at least 500.0 μM, at least 600.0 μM, at least 700.0 μM, at least 800.0 μM, at least 900.0 μM, or at least 1,000.0 μM.

As indicated above, the GLP-1/PYY secretion compositions of the invention comprise at least one of the natural receptor activating compounds and ligands referenced above that is adapted to bind to and activate at least olfactory receptor OR51E1, olfactory receptor OR1A1, olfactory receptor OR2C1 or olfactory receptor OR10J5.

The pharmacodynamic activity induced via activation of olfactory receptor OR51E1, olfactory receptor OR1A1, olfactory receptor OR2C1 and olfactory receptor OR10J5 is discussed in detail below.

### Olfactory Receptor OR51E1 Modulation

In a preferred embodiment of the invention, the GLP-1/PYY secretion compositions of the invention (and GLP-1/GIP secretion compositions of the invention, discussed below) comprise one or more natural receptor activating compounds and/or ligands that are specifically adapted to activate and, hence, modulate olfactory receptor OR51E1 activity.

As indicated above, activation of olfactory receptor OR51E1 induces a glucose-induced membrane depolarization of endogenous GI cells, more particularly, L-enteroendocrine (and, in some instances, pancreatic α-cells) and, thereby, opens the voltage-dependent Ca²⁺ (VDC) channels of the L-cells, whereby the resulting Ca²⁺ influx triggers vesicular exocytosis and increases secretion of GLP-1 and PYY from the cells.

The secreted GLP-1 binds to and activates GLP-1 receptor proteins on pancreatic β-cells, which induces secretion of insulin.

In a preferred embodiment, activation of olfactory receptor OR51E1 also effectuates secretion modulation and/or activation of additional endocrine factors, including, without limitation, ghrelin, gastrin, cholecystokinin (CCK), bombesin/gastrin releasing peptide (BBS/GRP), neurotensin (NT), glucagon-like peptide 2 (GLP-2), calcitonin gene-related peptide (CGRP), chromogranin A, enteroglucagon, galanin, leptin, motilin, amylin, neuropeptide Y (NPY), pancreatic polypeptide, substance P, oxyntomodulin and somatostatin.

In some embodiments of the invention, the preferred natural receptor activating compounds (and/or ligands) of the GLP-1/PYY secretion compositions (and GLP-1/GIP secretion compositions) of the invention, which are specifically adapted to activate olfactory receptor OR51E1 in vivo, comprise 3-methylpentanoic acid, 4-methylpentanoic acid, farnesol, eugenol, nonanoic acid, pentanol, butyl butyryl lactate and isovaleric acid.

### Olfactory Receptor OR1A1 Modulation

In some embodiments, the GLP-1/PYY secretion compositions (and GLP-1/GIP secretion compositions) of the invention comprise one or more receptor activating compounds and/or ligands that are specifically adapted to activate and, hence, modulate olfactory receptor OR1A1 activity, whereby pharmacodynamic activity similar to that induced via activation of olfactory receptor OR51E1 (discussed above) is induced.

In some embodiments of the invention, the preferred natural receptor activating compounds (and/or ligands) of the GLP-1/PYY secretion compositions (and GLP-1/GIP secretion compositions) of the invention, which are specifically adapted to activate olfactory receptor OR1A1 in vivo, comprise geraniol, citronellol, 3-methyl-2,4-nonanedione, estragole, neroli, heptanol, octanol, helional, nonanal, hydroxycitronellal and citral.

### Olfactory Receptor OR2C1 Modulation

In some embodiments, the GLP-1/PYY secretion compositions (and GLP-1/GIP secretion compositions) of the invention comprise one or more receptor activating compounds and/or ligands that are specifically adapted to activate and, hence, modulate olfactory receptor OR2C1 activity, whereby the following pharmacodynamic activity is induced.

Activation of olfactory receptor OR2C1 induces Ca²⁺ release from the endoplasmic reticulum of pancreatic β-cells through the phospholipase C-inositol triphosphate-dependent (PLC-IP3) pathway and, thereby, an increased concentration of intracellular Ca²⁺. The increase in intracellular Ca²⁺ then activates the CaMKK/CaMKIV pathway, which induces seminal insulinogenic processes, including glucokinase (GK) expression, and thereby glucose absorption by endogenous cells and glucose-stimulated insulin secretion (GSIS) from pancreatic islet cells.

The olfactory receptor OR2C1 thus indirectly induces GLP-1 production and secretion by endogenous GI cells via the above noted insulinogenic processes, which facilitate glucose-induced membrane depolarization of the GI cells induced by olfactory receptor activation (e.g., olfactory receptor OR51E1 activation) and, hence, secretion of GLP-1 from the GI cells resulting therefrom.

Activation of olfactory receptor OR2C1 similarly effectuates secretion modulation and/or activation of at least one of the aforementioned additional endocrine factors.

In some embodiments of the invention, the preferred natural receptor activating compounds (and/or ligands) of the GLP-1/PYY secretion compositions (and GLP-1/GIP secretion compositions) of the invention, which are specifically adapted to activate olfactory receptor OR2C1 in vivo, comprise octanoic acid, eugenol, musk ketone and (+)-dihydrocarvone.

### Olfactory Receptor OR10J5 Modulation

In some embodiments of the invention, the GLP-1/PYY secretion compositions (and GLP-1/GIP secretion compositions) of the invention comprise one or more receptor activating compounds and/or ligands that are specifically adapted to activate and, hence, modulate olfactory receptor OR10J5 activity, whereby the following pharmacodynamic activity is induced.

Activation of olfactory receptor OR10J5 induces downregulation of the seminal lipogenesis associated gene expression, including the expression of C/EBPα, PPARγ, RXR, LXRα, SREBP-1c, ap2, FAS, SCD1, ACC, and mtGPAT genes, and upregulation of mitochondrial and thermogenic gene expression, including the expression of PGC-1α, PRDM16, UCP1, Cytc, Cox4, and Cidea genes through the cAMP/PKA/HSL pathway.

The above noted downregulation of the seminal lipogenesis associated gene expression and upregulation of mitochondrial and thermogenic gene expression modulates lipid metabolism by inhibiting lipogenesis and, thus, reducing lipid accumulation in hepatic cells.

The olfactory receptor OR10J5 also indirectly induces GLP-1 production and secretion by endogenous GI cells via the above noted downregulation of the seminal lipogenesis associated gene expression, which restores normal lipogenic metabolic function and, thereby, facilitates glucose-induced membrane depolarization of the GI cells induced by olfactory receptor activation (e.g., olfactory receptor OR51E1 activation) and, hence, secretion of GLP-1 from the GI cells resulting therefrom.

In a preferred embodiment, the activation of olfactory receptor OR10J5 similarly effectuates secretion modulation and/or activation of at least one of the aforementioned additional endocrine factors.

In some embodiments of the invention, the preferred natural receptor activating compounds (and/or ligands) of the GLP-1/PYY secretion compositions (and GLP-1/GIP secretion compositions) of the invention, which are specifically adapted to activate olfactory receptor OR10J5 in vivo, similarly comprise α-cedrene, lyral and thujopsene.

### Olfactory Receptor-Mediated and Free Fatty Acid Receptor-Mediated (GIP) Secretion

In some embodiments of the invention, the natural receptor activating compounds and ligands of the invention, and compositions of the invention formed therefrom are specifically adapted to bind to and activate at least at least one receptor that induces GIP secretion in vivo including, without limitation, free fatty acid receptor FFAR1, free fatty acid receptor FFAR4, olfactory receptor OR2W1, olfactory receptor OR2B11, olfactory receptor OR2J3 and transient receptor TRPA1, whereby, as discussed in detail below, GIP secretion is induced in vivo.

The compositions formed from the noted natural receptor activating compounds and ligands are thus referred to herein as “GIP secretion compositions” (and, in some instances “GLP-1/GIP secretion compositions”).

According to the invention, the GIP secretion compositions of the invention (and GLP-1/GIP secretion compositions of the invention, discussed below), when delivered to a patient or subject, effectuate the following highly effective and, hence, desirable pharmacodynamic activity.

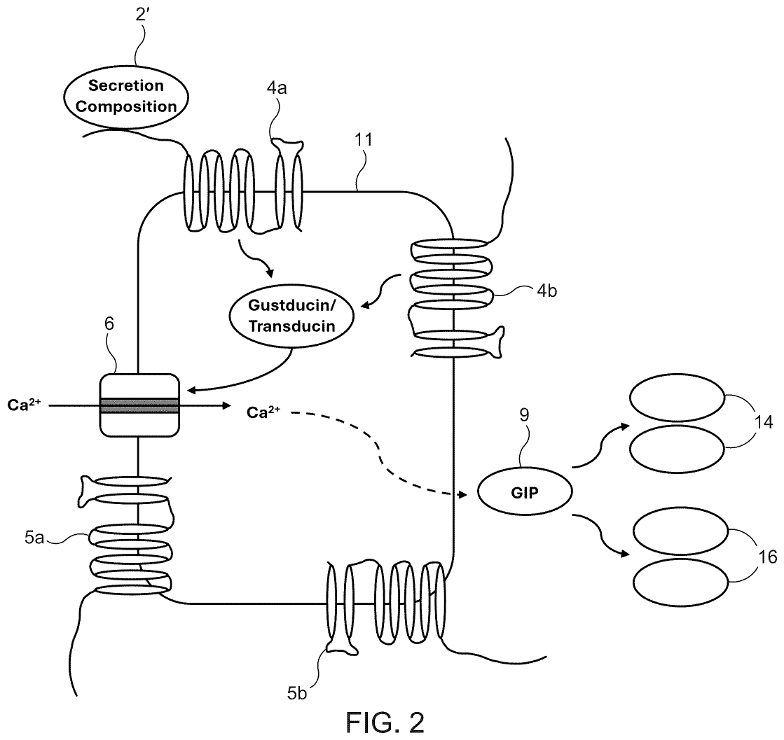

Referring to FIG. 2, when a GIP secretion composition of the invention (and GLP-1/GIP secretion composition of the invention) (denoted “2′”) is delivered to a subject or patient, the GIP secretion composition 2′ binds to at least one of the ectopic olfactory receptors referenced above (denoted “4a, 4b”) and at least one of the free fatty acid receptors (denoted “5a, 5b”) disposed on endogenous cells, in this instance, enteroendocrine K-cells, as depicted in FIG. 2, wherein a representative enteroendocrine K-cell is denoted “11”, whereby a gustducin-mediated cell signaling pathway is activated, which induces membrane depolarization of the enteroendocrine K-cell 11 (and, hence, cells) and opens the voltage-dependent Ca²⁺ (VDC) channels (denoted “6”) of the enteroendocrine K-cell(s) 11, wherein the resulting Ca²⁺ influx induces vesicular exocytosis and increased secretion of GIP (denoted “9”) from the enteroendocrine K-cell(s) 11.

As illustrated in FIG. 2, the secreted GIP 9 binds to and activates GIP receptor proteins on pancreatic β-cells (denoted “14”), which, induces secretion of insulin.

As further illustrated in FIG. 2, the secreted GIP 9 also binds to and activates GIP receptor proteins on endogenous GI cells (denoted “16”), such as islet cells of the pancreas, to promote pancreatic 3-cell survival and prevent apoptosis of pancreatic β-cells by activating the cAMP response element-binding (CREB) and Akt/PKB pathways, thus, directly, and indirectly maintaining a stable population of insulin-producing pancreatic β-cells in vivo.

As indicated above, the GIP secretion compositions (and GLP-1/GIP secretion compositions) of the invention comprise at least one receptor activating compound or ligand that is specifically adapted to bind to and activate at least free fatty acid receptor FFAR1, free fatty acid receptor FFAR4, olfactory receptor OR2W1, olfactory receptor OR2B11, olfactory receptor OR2J3 or transient receptor TRPA1.

As discussed below, in some embodiments, the preferred natural receptor activating compounds (and/or ligands) of the GIP secretion compositions (and GLP-1/GIP secretion compositions) comprise a medium-chain free fatty acid, including, without limitation, lauric acid, caproic acid, caprylic acid and capric acid; a long-chain free fatty acid, including, without limitation, palmitic acid and stearic acid; an omega-3 polyunsaturated acid, including, without limitation, alpha-linoleic acid, docosahexaenoic acid, and eicosatetraenoic acid; 2-heptanone, 1-octanal, (−)-citronellol, hexanal, 3-octanone, hexyl acetate, 1-hexanol, octanoic acid, 1-heptanol, allyl phenylacetate, benzyl acetate, 3,4-hexanedione, cis-3-hexen-1-ol, 2-ethyl-3,5-dimethylpyrazine, coumarin, dicyclohexyl disulfide, spearmint, coffee difuran, quinoline, cinnamaldehyde, allyl isothiocyanate, farnesyl thiosalicylic acid, formalin, hydrogen peroxide, 4-hydroxynonenal and acrolein.

According to the invention, the EC₅₀ values of the noted natural receptor activating compounds and ligands contained in a GIP secretion composition of the invention (and GLP-1/GIP secretion compositions of the invention) can similarly comprise any of the EC₅₀ values or EC₅₀ value ranges set forth above.

As indicated above, the natural receptor activating compounds and ligands of the GIP secretion compositions are specifically adapted to bind to and activate at least free fatty acid receptor FFAR1, free fatty acid receptor FFAR4, olfactory receptor OR2W1, olfactory receptor OR2B11, olfactory receptor OR2J3 or transient receptor TRPA1.

The pharmacodynamic activity induced via activation of free fatty acid receptor FFAR1, free fatty acid receptor FFAR4, olfactory receptor OR2W1, olfactory receptor OR2B11, olfactory receptor OR2J3 and transient receptor TRPA1 is discussed in detail below.

### Fatty Acid Receptor FFAR1 and FFAR4 Modulation

In some embodiments, the GIP secretion compositions of the invention (and GLP-1/GIP secretion compositions of the invention) comprise one or more receptor activating compounds and/or ligands that are specifically adapted to activate and, hence, modulate free fatty acid receptor FFAR1 and/or free fatty acid receptor FFAR4 activity, whereby the following pharmacodynamic activity is induced.

As indicated above, activation of free fatty acid receptors FFAR1 and FFAR4 induces membrane depolarization of enteroendocrine cells and opens voltage-dependent Ca²⁺ (VDC) channels of the enteroendocrine cells, wherein the resulting Ca²⁺ influx induces increased secretion of GIP from the enteroendocrine cells.

Activation of free fatty acid receptor FFAR1 and/or FFAR4, can, in some instances, also induce activation of G_{aq/11} and β-arrestin signaling pathways and, thereby, stimulate further GIP secretion.

As also indicated, the secreted GIP binds to and activates GIP receptor proteins on pancreatic β-cells, which induces secretion of insulin.

The secreted GIP also binds to and activates GIP receptor proteins on endogenous GI cells, such as islet cells of the pancreas, to promote pancreatic β-cell survival and prevent apoptosis of pancreatic β-cells by activating the cAMP response element-binding (CREB) and Akt/PKB pathways, thus, directly, and indirectly maintaining a stable population of insulin-producing pancreatic β-cells in vivo.

In a preferred embodiment, activation of free fatty acid receptor FFAR1 and/or FFAR4, also effectuates secretion modulation and/or activation of at least one of the aforementioned additional endocrine factors.

In some embodiments of the invention, the natural receptor activating compounds (and/or ligands) of the GIP secretion compositions (and GLP-1/GIP secretion compositions) of the invention, which are specifically adapted to activate free fatty acid receptor FFAR1 and/or FFAR4 in vivo, comprise a medium-chain free fatty acid, a long-chain free fatty acid, and an omega-3 polyunsaturated fatty acid.

In some embodiments, the GIP secretion compositions (and GLP-1/GIP secretion compositions) of the invention thus comprise lauric acid, caproic acid, caprylic acid, capric acid, palmitic acid, stearic acid, alpha-linoleic acid, docosahexaenoic acid and eicosatetraenoic acid.

### Olfactory Receptor OR2W1 Modulation

In some embodiments of the invention, the GIP secretion compositions (and GLP-1/GIP secretion compositions) of the invention comprise one or more receptor activating compounds and/or ligands that are specifically adapted to activate and, hence, modulate olfactory receptor OR2W1 activity, whereby the following pharmacodynamic activity is induced.

Activation of olfactory receptor OR2W1 by an OR2W1 activating compound and/or ligand induces a conformational change in the molecular structure of olfactory receptor OR2W1, which activates intracellular G_{α}/G_{β}/G_{γ} subunits of the receptor (See FIG. 1), whereby the subunits act as a guanine nucleotide exchange factor, wherein a guanine diphosphate (GDP) is exchanged for a guanine triphosphate (GTP), which binds to the Ga subunit of olfactory receptor OR2W1.

The noted binding of the GTP to the Ga subunit induces a dissociation of the G_{α}/G_{β}/G_{γ} subunits of olfactory receptor OR2W1 into a (i) free Ga subunit, which binds to adenyl cyclase (AC) III (See FIG. 1), and a (ii) G_{β}/G_{γ} complex (See also FIG. 1) and, thereby, activates seminal downstream cell signaling processes that induce an increase in intracellular cAMP in endogenous cells, such as enteroendocrine cells.

As depicted in FIG. 2, by virtue of the intracellular cAMP level increase, among other cell signaling processes, opening of cyclic nucleotide gated Ca²⁺ channels is induced, which results in increased cellular Ca²⁺ and, thereby, increased induction of secretion of GIP from the endogenous cells.

The secreted similarly GIP binds to and activates GIP receptor proteins on pancreatic β-cells, which, as indicated above, (i) induces secretion of insulin, (ii) promotes pancreatic 3-cell survival, and (iii) prevents apoptosis of pancreatic β-cells by activating the cAMP response element-binding (CREB) and Akt/PKB pathways, thus, directly and indirectly maintaining a stable population of insulin-producing pancreatic β-cells in vivo.

In some embodiments, the activation of olfactory receptor OR2W1 similarly effectuates secretion modulation and/or activation of at least one of the aforementioned additional endocrine factors.

In some embodiments of the invention, the preferred natural receptor activating compounds (and/or ligands) of the GIP secretion compositions (and GLP-1/GIP secretion compositions), which are adapted to activate OR2W1 in vivo comprise 2-heptanone, 1-octanal, (−)-citronellol, hexanal, 3-octanone, hexyl acetate, 1-hexanol, octanoic acid, 1-heptanol, allyl phenylacetate, benzyl acetate, 3,4-hexanedione and cis-3-hexen-1-ol.

### Olfactory Receptor OR2B11 Modulation

In some embodiments of the invention, the GIP secretion compositions (and GLP-1/GIP secretion compositions) of the invention comprise one or more receptor activating compounds and/or ligands that are specifically adapted to activate and, hence, modulate olfactory receptor OR2B11 activity, whereby pharmacodynamic activity similar to that induced via activation of olfactory receptor OR2W1 (discussed above) is induced.

In some embodiments of the invention, the preferred natural receptor activating compounds (and/or ligands) of the GIP secretion compositions (and GLP-1/GIP secretion compositions) of the invention, which are adapted to activate OR2B11 in vivo comprise 2-ethyl-3,5-dimethylpyrazine, coumarin, dicyclohexyl disulfide, spearmint, coffee difuran, quinoline and cinnamaldehyde.

### Olfactory Receptor OR2J3 Modulation

In some embodiments of the invention, the GIP secretion compositions of the invention (and GLP-1/GIP secretion compositions of the invention) thus comprise one or more receptor activating compounds and/or ligands that are specifically adapted to activate and, hence, modulate olfactory receptor OR2J3 activity, whereby pharmacodynamic activity similar to that induced via activation of olfactory receptor OR2W1 (discussed above) is induced.

In some embodiments of the invention, the preferred natural receptor activating compounds (and/or ligands) of the GIP secretion compositions (and GLP-1/GIP secretion compositions), which are adapted to activate, which are adapted to activate OR2J3 in vivo comprise cis-3-hexen-1-ol and cinnamaldehyde.

### Transient Receptor TRPA1 Modulation

In some embodiments of the invention, the GIP secretion compositions (and GLP-1/GIP secretion compositions) of the invention comprise one or more receptor activating compounds and/or ligands that are specifically adapted to activate and, hence, modulate transient receptor TRPA1 activity, whereby the following pharmacodynamic activity is induced.

It is believed that, when transient receptor TRPA1 is activated, the compound cellular Ca²⁺ is increased, whereby serotonin (5-HT) secretion from endogenous enterochromaffin cells is increased.

The serotonin secreted from the enterochromaffin cells binds to 5-HT receptors of endogenous gastrointestinal cells and, thereby, induces GIP secretion by the endogenous cells.

In some embodiments of the invention, the preferred natural receptor activating compounds (and/or ligands) of the GIP secretion compositions (and GLP-1/GIP secretion compositions) of the invention, which are adapted to activate transient receptor TRPA1 in vivo, comprise allyl isothiocyanate, cinnamaldehyde, farnesyl thiosalicylic acid, formalin, hydrogen peroxide, 4-hydroxynonenal and acrolein.

In some embodiments, the GIP secretion compositions (and GLP-1/GIP secretion compositions) of the invention are formulated and adapted to activate and, hence, modulate at least one olfactory receptor; specifically, olfactory receptor OR51E1, olfactory receptor OR2C1 or olfactory receptor OR2W1, and at least one free fatty acid receptor; specifically, free fatty acid receptor FFAR1 or free fatty acid receptor FFAR4, and, in some instances, at least one transient receptor; specifically, transient receptor TRPA1 in vivo when delivered to a patient.

In a preferred embodiment, the GIP secretion compositions of the invention and, as discussed below, GLP-1/GIP secretion compositions of the invention, are adapted to induce at least 50% activation of at least olfactory receptor OR2W1 and/or olfactory receptor OR2B11 and/or OR2J3, free fatty acid receptor FFAR1 and/or free fatty acid receptor FFAR4, and/or transient receptor TRPA1 in vivo when delivered to a patient.

### Concomitant Modulation of GLP-1 and GIP Secretion

In some embodiments of the invention, the natural insulin modulating compounds and ligands, and compositions of the invention formed therewith, are specifically adapted to bind to and activate and, hence, modulate at least one insulin modulating receptor that induces GLP-1 secretion and at least one insulin modulating receptor that induces GIP secretion in vivo, including, without limitation, olfactory receptor OR51E1, olfactory receptor OR1A1, olfactory receptor OR2C1, olfactory receptor OR10J5, olfactory receptor OR2W1, olfactory receptor OR2B11, olfactory receptor OR2J3, free fatty acid receptor FFAR1, free fatty acid receptor FFAR4 and transient receptor TRPA1, whereby the pharmacodynamic activity associated with the selective insulin modulating receptor described above is induced.

The compositions formed from the noted natural receptor activating compounds and ligands are thus referred to herein as “GLP-1/GIP secretion compositions.”

According to the invention, GLP-1/GIP secretion compositions can thus comprise one or more of the natural receptor activating compounds and ligands referenced above.

In a preferred embodiment, the natural receptor activating compounds of the GLP-1/GIP secretion compositions of the invention comprise 3-methylpentanoic acid, 4-methylpentanoic acid, farnesol, eugenol, nonanoic acid, pentanol, butyl butyryl lactate, isovaleric acid, geraniol, citronellol, 3-methyl-2,4-nonanedione, estragole, neroli, heptanol, octanol, helional, nonanal, hydroxycitronellal, citral, octanoic acid, musk ketone, (+)-dihydrocarvone, α-cedrene, lyral, thujopsene, lauric acid, caproic acid, caprylic acid, capric acid, palmitic acid, stearic acid, alpha-linoleic acid, docosahexaenoic acid, eicosatetraenoic acid, 2-heptanone, 1-octanal, (−)-citronellol, hexanal, 3-octanone, hexyl acetate, 1-hexanol, octanoic acid, 1-heptanol, allyl phenylacetate, benzyl acetate, 3,4-hexanedione, cis-3-hexen-1-ol, 2-ethyl-3,5-dimethylpyrazine, coumarin, dicyclohexyl disulfide, spearmint, coffee difuran, quinoline, cinnamaldehyde, allyl isothiocyanate, farnesyl thiosalicylic acid, formalin, hydrogen peroxide, 4-hydroxynonenal and acrolein.

As indicated above, according to the invention, the EC₅₀ value of the noted natural receptor activating compounds and ligands contained in a GLP-1/GIP secretion composition of the invention can comprise any of the aforementioned EC₅₀ value ranges and EC₅₀ values therebetween.

As indicated above, in a preferred embodiment, the GLP-1/GIP secretion compositions of the invention are formulated and adapted to activate and, hence, modulate at least one of the insulin modulating receptors of the invention, i.e., olfactory receptor OR51E1, olfactory receptor OR1A1, olfactory receptor OR2C1, olfactory receptor OR10J5, olfactory receptor OR2W1, olfactory receptor OR2B11, olfactory receptor OR2J3, free fatty acid receptor FFAR1, free fatty acid receptor FFAR4 and transient receptor TRPA1.

In some embodiments, the GLP-1/GIP secretion compositions of the invention are specifically formulated and adapted to activate at least olfactory receptor OR51E1 and free fatty acid receptor FFAR1.

In a preferred embodiment, the GLP-1/GIP secretion compositions of the invention are adapted to induce at least 50% activation of at least olfactory receptor OR51E1 and/or olfactory receptor OR1A1 and/or olfactory receptor OR2W1 and/or olfactory receptor OR2B11, free fatty acid receptor FFAR1 and/or free fatty acid receptor FFAR4, and/or transient receptor TRPA1 in vivo when delivered to a patient.

As indicated above, in a preferred embodiment, the GLP-1/GIP secretion compositions of the invention are adapted to induce at least 50% activation of multiple receptors; particularly, olfactory receptor OR51E1 and free fatty acid receptor FFAR1 or free fatty acid receptor FFAR4.

According to the invention, modulating the activity of multiple receptors, e.g., olfactory receptors and/or free fatty acid receptors and/or transient receptor potential ion channels, as described herein, results in elevated endocrine factor levels.

In some embodiments, modulating the activity of multiple receptors, e.g., olfactory receptors and/or free fatty acid receptors and/or transient receptor potential ion channels, as described herein, results in synergistically elevated endocrine factor levels.

In some embodiments, modulating the activity of multiple receptors, e.g., olfactory receptors and/or free fatty acid receptors and/or transient receptor potential ion channels, as described herein, results in elevated endocrine factor secretion.

In some embodiments, modulating the activity of multiple receptors, e.g., olfactory receptors and/or free fatty acid receptors and/or transient receptor potential ion channels, as described herein, results in synergistically elevated endocrine factor secretion.

In some embodiments, modulating the activity of multiple receptors, e.g., olfactory receptors and/or free fatty acid receptors and/or transient receptor potential ion channels, as described herein, results in endocrine factor secretion higher than endocrine factor secretion induced when modulating the activity of any single receptor alone.

In some embodiments, the GLP-1/PYY, GIP and GLP-1/GIP secretion compositions of the invention further comprise a physiologically suitable (or acceptable) carrier (also referred to herein as a physiologically suitable (or acceptable) excipient, or physiologically suitable (or acceptable) excipient selected based on a chosen route of administration, e.g., oral administration, and standard pharmaceutical practice.

According to the invention, suitable aqueous and non-aqueous carriers that can be employed in the secretion compositions of the invention include water, ethanol, polyols (such as glycerol, glycerin-based water, propylene glycol, polyethylene glycol, and the like), and suitable mixtures thereof; vegetable oils, such as olive oil; buffers, such as neutral buffered saline, phosphate buffered saline and the like; carbohydrates, such as glucose, mannose, sucrose, and dextran, mannitol; proteins; polypeptides, and amino acids, such as glycine; antioxidants; chelating agents such as ethylenediaminetetraacetic acid (EDTA) or glutathione; adjuvants (e.g., aluminum hydroxide); and injectable organic esters, such as ethyl oleate and cyclodextrins.

According to the invention, the GLP-1/PYY, GIP and GLP-1/GIP secretion compositions of the invention can be manufactured by means of conventional mixing, dissolving, granulating, dragee-making, levigating, emulsifying, encapsulating, entrapping, and lyophilizing processes. The manufactured compositions can take the form of solutions, suspensions, emulsion, tablets, pills, pellets, capsules, capsules containing liquids, powders, sustained-release formulations, suppositories, emulsions, aerosols, sprays, suspensions, and other forms suitable for administration to a patient.

In some embodiments, proper fluidity of a composition is maintained via coating materials, such as lecithin.

According to the invention, the GLP-1/PYY, GIP and GLP-1/GIP secretion compositions of the invention can be formulated into any known form suitable for parenteral administration, e.g., injection or infusion. Alternatively, the compositions can be formulated for oral administration, nasal or other mucosal tissue administration, or administration as a suppository (e.g., for small molecules). The compositions can also comprise formulation additives, such as suspending agents, preservatives, stabilizers and/or dispersants, and preservation agents.

According to the invention, the GLP-1/PYY, GIP and GLP-1/GIP secretion compositions can thus be administered to a patient via any suitable method, including, without limitation, oral, sublingual, inhalation, intranasal, epidural, intracerebral, transdermal, topical, and injection administration means.

The GLP-1/PYY, GIP and GLP-1/GIP secretion compositions of the invention can also be administered to a patient via intraarterial, subcutaneous, intradermal, intratumoral, intranodal, intramedular, intramuscular, intranasally, and intraperitoneal means.

According to the invention, the GLP-1/PYY, GIP and GLP-1/GIP secretion compositions of the invention can also be incorporated into various ingestible fluids, such as flavored waters and coffee.

According to the invention, the GLP-1/PYY, GIP and GLP-1/GIP secretion compositions of the invention can also be incorporated into a food item, such as a cracker, and/or a nutritional supplement or supplemental food item, such as protein bar.

According to the invention, the GLP-1/PYY, GIP and GLP-1/GIP secretion compositions of the invention can be administered at any of the following dosage ranges: from about 1.0 g/kg to about 1.0 kg/kg, about 10.0 g/kg to about 100.0 g/kg, about 10.0 g/kg to about 25.0 mg/kg, about 100.0 g/kg to about 50.0 g/kg, about 100.0 g/kg to about 50.0 mg/kg, about 500.0 g/kg to about 25.0 g/kg, about 500.0 g/kg to about 100.0 mg/kg, about 1.0 mg/kg to about 10.0 g/kg, about 1.0 mg/kg to about 50.0 mg/kg, about 5.0 mg/kg to about 5.0 g/kg, about 5.0 mg/kg to about 25.0 mg/kg, about 10.0 mg/kg to about 2.5 g/kg, about 10.0 mg/kg to about 200.0 mg/kg, about 25.0 mg/kg to about 1.5 g/kg, about 25.0 mg/kg to about 750.0 mg/kg, about 50.0 mg/kg to about 1.0 g/kg, about 50.0 mg/kg to about 600.0 mg/kg, about 75.0 mg/kg to about 550.0 mg/kg, about 100.0 mg/kg to about 500.0 mg/kg, about 150.0 mg/kg to about 400.0 mg/kg, and about 200.0 mg/kg to about 350.0 mg/kg.

The GLP-1/PYY, GIP and GLP-1/GIP secretion compositions of the invention can also be administered at any dosage between the above referenced dosage ranges.

The GLP-1/PYY, GIP and GLP-1/GIP secretion compositions of the invention can thus also be administered at a dosage of at least about 1.0 g/kg, at least about 10.0 g/kg, at least about 100.0 g/kg, at least about 500.0 g/kg, at least about 1.0 mg/kg, at least about 5.0 mg/kg, at least about 10.0 mg/kg, at least about 25.0 mg/kg, at least about 50.0 mg/kg, at least about 75.0 mg/kg, at least about 100.0 mg/kg, at least about 150.0 mg/kg, and at least about 200.0 mg/kg.

According to the invention, GLP-1/PYY, GIP and GLP-1/GIP secretion compositions of the invention can also be administered at one of the dosage ranges (and/or dosages therebetween) over a prescribed time, by way of example, from about 1.0 g to about 1.0 kg per day, from about 100.0 g to about 500.0 g per day, from about 500.0 g to about 100.0 g per day, from about 1.0 mg to about 20.0 g per day, from about 2.5 mg to about 15.0 g per day, from about 5.0 mg to about 10.0 g per day, from about 10.0 mg to about 5.0 g per day, from about 25.0 mg to about 2.5 g per day, from about 50.0 mg to about 2.0 g per day, from about 100.0 mg to about 1.5 g per day, from about 150.0 mg to about 1.0 g per day, from about 200.0 mg to about 750.0 mg per day, and from about 250.0 mg to about 500.0 mg per day.

According to the invention, the noted dosages and delivery protocols are sufficient to induce sustained (i.e., extended periods) of GLP-1, PYY and/or GIP secretion in vivo.

As indicated above, the GLP-1/PYY, GIP and GLP-1/GIP compositions of the invention, when delivered to a patient, effectively and safely modulate, i.e., increase, insulin and PYY secretion and, hence, can be readily employed to treat cardiovascular diseases and disorders, as well as a multitude of other physiological disorders associated with (or caused by) abnormal insulin secretion (and insulin resistance) (referred to herein as “insulin-resistance-induced physiological disorders); particularly, type 2 diabetes mellitus and obesity.

Indeed, as discussed in detail below, recent studies reflect that insulin resistance and, hence, hyperglycemia, which is often associated therewith, is also associated with various cardiovascular disorders, such as atherosclerosis, by virtue of vascular endothelial dysfunction, vascular smooth muscle cell (VSMC) over-proliferation and migration, and monocyte/macrophage/foam cell accumulation associated therewith. See Austin, et al., Hypertriglyceridemia as a Cardiovascular Risk, American Journal of Cardiology, v. 81(4), pp. 7B-12B (1998).

As also indicated above, recent studies also reflect that there is a strong correlation between abnormal insulin secretion (and insulin resistance-associated) disorders; particularly, type 2 diabetes mellitus, and cardiovascular disorders.

Indeed, it is established that type 2 diabetes mellitus often accompanies hypertriglyceridemia characterized by a lipid triad, which includes (i) high serum levels of plasma triglycerides, (ii) low serum levels of high-density lipoproteins (HDLs), and (iii) high serum levels of low-density lipoproteins (LDLs) in the individual's blood. Type 2 diabetes mellitus is also often characterized by excessive postprandial lipemia, i.e., abnormally high serum triglyceride levels after meal consumption. See Goldberg, Ira J., Diabetic Dyslipidemia: Causes and Consequences, Journal of Clinical Endocrinology & Metabolism, v. 86(3), pp. 965-971 (2001).

It is also well established that there is a strong correlation between obesity and type 2 diabetes mellitus (and, hence, cardiovascular disorders). Indeed, recent research confirms that obesity, i.e., excess visceral fat mass, increases adiposity and results in hypertriglyceridemia, whereby, adipocytes release chemotactic factors, such as monocyte chemoattractant protein-1 (MCP-1) and tumor necrosis factor-α (TNFα), which modulate inflammatory responses in adipose tissue. MCP-1 initiates the migration of monocytes into visceral adipose tissue (VAT) and promotes their differentiation into mature macrophages.

The mature macrophages then secrete large amounts of pro-inflammatory cytokines, e.g., TNFα and IL-1β, and, thereby, (i) increase lipolysis, (ii) decrease insulin-stimulated glucose transporter type-4 (GLUT4) glucose transport in muscle tissue, and (iii) impair triglyceride biosynthesis and adipocyte storage in VAT, which results in an increase in circulating serum triglyceride and oxidized LDL (oxLDL) levels and, thereby, ectopic lipid deposition of toxic fatty acid species (e.g., diacylglycerol and ceramide) in extra-adipose tissue, such as the pancreas. The ectopic lipid deposition of the fatty acid species results in insufficient insulin production and secretion by the pancreas via pancreatic β-cell impairment and insulin resistance by inhibiting insulin-stimulated glucose transport in muscle tissue through activation of protein kinases protein kinase C (PKC), IKKβ, and JNK, which can, and in many instances will, result in type 2 diabetes mellitus. See Guilherme, et al., Adipocyte Dysfunctions Linking Obesity to Insulin Resistance and Type 2 Diabetes, Nature Reviews: Molecular cell Biology, v. 9.5, pp. 367-377 (2008).

As indicated above, in a preferred embodiment, the GLP-1/PYY, GIP and GLP-1/GIP compositions of the invention can, and will, effectively and safely induce secretion of GLP-1 and, thereby, insulin secretion in vivo when delivered to a patient.

According to the invention, the GLP-1/PYY, GIP and GLP-1/GIP compositions of the invention are also adapted to reduce the hepatic secretion of glucose and, thereby, abate hyperglycemia and, thereby, increase systemic insulin sensitivity when delivered to a patient.

As discussed in detail in Applicant's priority U.S. application Ser. No. 18/430,796, which is incorporated by reference herein in its entirety, the GLP-1 secreted by the GLP-1/PYY, GIP and GLP-1/GIP compositions of the invention also decreases the rate of gastric emptying and acid secretion, resulting in reduced appetite and, thereby, induced weight loss. The weight loss also ameliorates hypertriglyceridemia, hyperglycemia and increases systemic insulin sensitivity.

As also indicated above and discussed in detail in priority U.S. application Ser. No. 18/430,796, the GLP-1/PYY and GLP-1/GIP compositions of the invention, when delivered to a patient, also effectively and safely induce secretion of PYY in vivo, which promotes satiety and also decreases the rate of gastric emptying, whereby weight loss and, thereby, increased systemic insulin sensitivity are further induced and hypertriglyceridemia and hyperglycemia is further ameliorated.

The GLP-1/PYY, GIP and GLP-1/GIP compositions of the invention, when delivered to a patient presenting with a cardiovascular disorder; particularly, atherosclerosis, diabetic cardiomyopathy or hypertension, are thus adapted to effectively and safely treat the cardiovascular disorder, and ameliorate at least one seminal physiological risk factor associated with the cardiovascular disorder and/or at least one pathophysiological effect associated with the cardiovascular disorder.

The GLP-1/PYY, GIP and GLP-1/GIP compositions of the invention, when delivered to a patient presenting with type 2 diabetes mellitus, are thus also adapted to effectively and safely treat the type 2 diabetes mellitus, and ameliorate at least one seminal physiological risk factor associated with the type 2 diabetes mellitus and/or at least one pathophysiological effect induced by the type 2 diabetes mellitus.

The GLP-1/PYY, GIP and GLP-1/GIP compositions of the invention, when delivered to a patient presenting with obesity, are thus also adapted to effectively and safely treat the obesity, and ameliorate at least one seminal physiological risk factor associated with the obesity and/or at least one pathophysiological effect induced by the obesity.

The GLP-1/PYY, GIP and GLP-1/GIP compositions of the invention, when delivered to a patient presenting with a cardiovascular disorder and type 2 diabetes mellitus, are also adapted to effectively and safely treat the cardiovascular disorder and the type 2 diabetes mellitus, and ameliorate at least one seminal physiological risk factor associated with the cardiovascular disorder and/or at least one pathophysiological effect associated with cardiovascular disorder, and ameliorate at least one seminal physiological risk factor associated with the type 2 diabetes mellitus and/or at least one pathophysiological effect associated with the type 2 diabetes mellitus.

The GLP-1/PYY, GIP and GLP-1/GIP compositions of the invention, when delivered to a patient presenting with a cardiovascular disorder, obesity and type 2 diabetes mellitus, are also adapted to effectively and safely treat the cardiovascular disorder, obesity and type 2 diabetes mellitus, and ameliorate at least one seminal physiological risk factor associated with the cardiovascular disorder and/or at least one pathophysiological effect associated with the cardiovascular disorder, ameliorate at least one seminal physiological risk factor associated with the obesity and/or at least one pathophysiological effect associated with the obesity, and ameliorate at least one seminal physiological risk factor associated with the type 2 diabetes mellitus and/or at least one pathophysiological effect associated with the type 2 diabetes mellitus.

According to the invention, the GLP-1/PYY, GIP and GLP-1/GIP compositions of the invention are also adapted to treat a cardiovascular disorder presented by a patient, and ameliorate at least one seminal physiological risk factor associated with the cardiovascular disorder and/or at least one pathophysiological effect associated with the cardiovascular disorder, without the patient also presenting with obesity or type 2 diabetes mellitus.

Treatment of three (3) seminal cardiovascular disorders, i.e., atherosclerosis, diabetic cardiomyopathy and hypertension, with the GLP-1/PYY, GIP and GLP-1/GIP secretion compositions of the invention is discussed in detail below.

### Atherosclerosis

Atherosclerosis is a chronic immunoinflammatory, fibroproliferative disease in which there is a build-up of plaques inside arteries. The plaques are commonly referred to as atheromas or atherosclerotic plaques.

The noted plaques are principally composed of lipids and can, and in many instances will, induce acute, life-threatening cardiovascular events, such as angina pectoris and cerebral ischemia, and, thereby, severe cardiovascular complications, such as heart failure.

As discussed in detail below, in some embodiments of the invention, the GLP-1/PYY, GIP and GLP-1/GIP compositions are adapted to effectively and safely (i) treat atherosclerosis, (ii) ameliorate and/or stabilize at least one physiological risk factor associated with atherosclerosis, and (iii) preferably, ameliorate and/or stabilize at least one pathophysiological effect associated with atherosclerosis, when the GLP-1/PYY, GIP and GLP-1/GIP compositions are delivered to a patient.

Indeed, according to the invention, in some embodiments, when the GLP-1/PYY, GIP and GLP-1/GIP compositions of the invention are delivered to a patient presenting with atherosclerosis, the GLP-1/PYY, GIP and GLP-1/GIP compositions effectively and safely treat the atherosclerosis, i.e., inhibit the formation and progression of the atherosclerosis, and ameliorate at least one physiological risk factor associated with atherosclerosis and/or at least one seminal pathophysiological effect associated with atherosclerosis by, among other physiological processes, (i) reducing lipid deposition into muscle tissue by increasing systemic insulin sensitivity, (ii) activating endothelial nitric oxide synthase (eNOS), (iii) activating the AMP-activated protein kinase (AMPK) signaling pathway to inhibit LDL synthesis and proinflammatory processes, (iv) inducing seminal vascular anti-inflammatory processes; particularly, suppression of foam cell formation by activating GLP-1 receptor signaling induced autophagy, reduction of ACAT1 expression/activity, and inhibition of the PKA/CD36 pathway to inhibit oxLDL uptake and accumulation by macrophages, (v) promoting β-cell proliferation in pancreatic tissue, and (vi) ameliorating, and in some instances, reversing, maladaptive vascular remodeling (such as neointimal hyperplasia) and luminal stenosis resulting therefrom, after vascular injury by suppressing vascular smooth muscle cell (VSMC) over-proliferation and migration via the cAMP/PKA pathway, and endothelial dysfunction via apoptosis of vascular endothelial and smooth muscle cells.

In some embodiments of the invention, the GLP-1/PYY, GIP and GLP-1/GIP compositions of the invention also delay the progression of the atherosclerosis, and ameliorate at least one physiological risk factor associated with atherosclerosis and/or at least one seminal pathophysiological effect associated with atherosclerosis by attenuating monocyte/macrophage/foam cell accumulation proximate arterial walls by inhibiting the adhesion of the monocytes to activated endothelium, i.e., endothelium characterized by a proinflammatory and procoagulation state, of the arteries and the transformation of the macrophages into foam cells.

In some embodiments, the GLP-1/PYY, GIP and GLP-1/GIP secretion compositions of the invention thus restrict the formation of atherosclerotic plaques and stabilize the development thereof and, hence, abate vascular inflammation resulting from the atherosclerotic plaques via activation of the AMPK signaling pathway, induced vascular anti-inflammatory processes and suppression of VSMC over-proliferation and migration.

Activation of the AMPK signaling pathway also decreases the secretion of pro-inflammatory and pro-apoptotic cytokines, which induce apoptosis of vascular endothelial and smooth muscle cells in a vessel.

Thus, according to the invention, in some embodiments, the GLP-1/PYY, GIP and GLP-1/GIP compositions of the invention are adapted to effectively and safely treat atherosclerosis presented by a patient, i.e., inhibit the formation of atherosclerotic plaques and progression of the atherosclerosis, by inducing multiple seminal physiological processes, including, without limitation, (i) increasing systemic insulin sensitivity, (ii) activating the AMPK signaling pathway, (iii) promoting β-cell proliferation, (iv) suppressing VSMC over-proliferation and migration after vascular injury, and (v) inducing seminal vascular anti-inflammatory processes, e.g., suppressing foam cell activation, attenuating monocyte/macrophage/foam cell accumulation proximate arterial walls, reducing ACAT1 expression/activity, and inhibiting the PKA/CD36 pathway.

As indicated above, according to the invention, the noted GLP-1/PYY, GIP and GLP-1/GIP compositions are also adapted to ameliorate at least one physiological risk factor associated with the atherosclerosis, including, without limitation, systemic insulin resistance, impaired insulin production, diminished NO presence in the extracellular space proximate vascular endothelial cells, and vascular inflammation.

As also indicated above, the noted GLP-1/PYY, GIP and GLP-1/GIP compositions of the invention are also preferably adapted to ameliorate at least one pathophysiological effect associated with atherosclerosis, including, without limitation, endothelial dysfunction, e.g., apoptosis of vascular endothelial and smooth muscle cells, vascular inflammation, maladaptive vascular remodeling (including vascular fibrosis and intimal neoplasia) and luminal stenosis.

Thus, in some embodiments of the invention, the GLP-1/PYY, GIP and GLP-1/GIP compositions of the invention, when delivered to a patient presenting with atherosclerosis, can, and will, effectively and safely (i) treat atherosclerosis, (ii) ameliorate and/or stabilize at least one physiological risk factor of atherosclerosis, and (iii) preferably ameliorate and/or stabilize at least one pathophysiological effect associated with atherosclerosis.

As indicated above, recent studies reflect that there is also a strong correlation between insulin-resistance-induced physiological disorders; particularly, type 2 diabetes mellitus, and, hence, the physiological risk factors associated therewith, and atherosclerosis. See, e.g., Goldberg, Ira J., Diabetic Dyslipidemia: Causes and Consequences, Journal of Clinical Endocrinology & Metabolism, v. 86(3), pp. 965-971 (2001); Ormazabal, et al., Association between Insulin Resistance and the Development of Cardiovascular Disease, Cardiovascular Diabetology, v.17, pp. 1-14 (2018); Semenkovich, C. F., Insulin Resistance and Atherosclerosis, Journal of Clinical Investigation, v. 116(7), pp. 1813-1822 (2006); and O'Leary, et al., Insulin Sensitivity and Atherosclerosis, The Insulin Resistance Atherosclerosis Study (IRAS) Investigators, Circulation, v. 93(10), pp. 1809-17 (1996).

Indeed, as indicated above, the studies reflect that insulin resistance (even at an early stage) induces a proatherogenic lipid phenotype in a pre-diabetic individual. In the initial stages of systemic insulin resistance, serum free fatty acid (FFA) levels are increased due to a decreased suppression of lipolysis in adipocytes. Systemic insulin resistance also abates degradative pathways for apolipoprotein B (apoB) in hepatic tissue, which results in increased LDL production and, hence, increased serum levels of LDL.

The studies further reflect that systemic insulin resistance decreases the activity of lipoprotein lipase, which is found in the endothelium of peripheral capillaries and is a rate-limiting factor for the clearance of triglyceride-dense lipoproteins in an insulin-resistant individual. The decreased activity of lipoprotein lipase results in hypertriglyceridemia, which, as discussed above, is characterized by a lipid triad, including (i) high serum levels of plasma triglycerides, (ii) low serum levels of HDLs, and (iii) high serum levels of LDLs in the individual's blood.

The LDLs in the individual's blood then oxidize into oxLDLs that induce the immunogenic release of chemotactic factors from adipocytes that modulate inflammatory responses in adipose tissue, such as MCP-1 and TNFα.

As further indicated above, MCP-1 initiates the migration of monocytes into visceral adipose tissue (VAT) and promotes their differentiation into mature macrophages. The mature macrophages then secrete large amounts of pro-inflammatory cytokines and, thereby, (i) increase lipolysis, (ii) decrease GLUT4-mediated glucose transport in muscle tissue, and (iii) impair triglyceride biosynthesis and adipocyte storage in VAT, which results in a further increase in circulating serum triglyceride levels and, thereby, ectopic lipid deposition of toxic fatty acid species in extra-adipose tissue.

The mature macrophages also uptake and accumulate circulating oxLDL in an individual's blood and, thereby, transform into foam cells that adhere to the individual's arterial walls and become the foundation of an atheroma or an atherosclerotic plaque.

There is, thus, a strong correlation between the physiological risk factors associated with insulin-resistance-induced physiological disorders; particularly, type 2 diabetes mellitus, and atherosclerosis.

As indicated above, there is also a strong correlation between obesity and type 2 diabetes mellitus. See, e.g., Guilherme, et al., Adipocyte Dysfunctions Linking Obesity to Insulin Resistance and Type 2 Diabetes, Nature Reviews: Molecular Cell Biology, v. 9.5, pp. 367-377 (2008).

Indeed, as also indicated above, recent research confirms that obesity, i.e., excess visceral fat mass, increases adiposity and results in hypertriglyceridemia, whereby, adipocytes release chemotactic factors, such as monocyte chemoattractant protein-1 (MCP-1) and tumor necrosis factor-α (TNFα), which modulate inflammatory responses in adipose tissue. MCP-1 initiates the migration of monocytes into visceral adipose tissue (VAT) and promotes their differentiation into mature macrophages.

The mature macrophages then secrete large amounts of pro-inflammatory cytokines, e.g., TNFα and IL-1β, and, thereby, (i) increase lipolysis, (ii) decrease insulin-stimulated glucose transporter type-4 (GLUT4) glucose transport in muscle tissue, and (iii) impair triglyceride biosynthesis and adipocyte storage in VAT, which results in an increase in circulating serum triglyceride and oxidized LDL (oxLDL) levels and, thereby, ectopic lipid deposition of toxic fatty acid species (e.g., diacylglycerol and ceramide) in extra-adipose tissue, such as the pancreas. The ectopic lipid deposition of the fatty acid species results in insufficient insulin production and secretion by the pancreas via pancreatic β-cell impairment and insulin resistance by inhibiting insulin-stimulated glucose transport in muscle tissue through activation of protein kinases protein kinase C (PKC), IKKβ, and JNK, which can, and in many instances will, result in type 2 diabetes mellitus.

As indicated above, in some embodiments of the invention, when the GLP-1/PYY, GIP and GLP-1/GIP compositions of the invention are delivered to a patient presenting with atherosclerosis, the GLP-1/PYY, GIP and GLP-1/GIP compositions of the invention can, and will, effectively and safely (i) treat atherosclerosis by restricting the formation of atherosclerotic plaques and stabilizing and, hence, restricting the progression of the atherosclerotic plaques, and (ii) ameliorate and/or stabilize at least one physiological risk factor of atherosclerosis, and (iii) preferably ameliorate and/or stabilize at least one pathophysiological effect associated with atherosclerosis.

As set forth in Applicant's priority U.S. application Ser. Nos. 18/430,796 and 18/980,129, when the GLP-1/PYY, GIP and GLP-1/GIP compositions of the invention are delivered to a patient presenting with obesity and/or type 2 diabetes mellitus, the GLP-1/PYY, GIP and GLP-1/GIP compositions of the invention can, and will, also effectively and safely treat the obesity and/or treat type 2 diabetes mellitus, and ameliorate at least one physiological risk factor associated with type 2 diabetes mellitus.

Indeed, as indicated above and set forth in Applicant's priority U.S. application Ser. No. 18/430,796, the GLP-1 secretion induced by the GLP-1/PYY, GIP and GLP-1/GIP compositions of the invention decreases the rate of gastric emptying and acid secretion, resulting in reduced appetite and, thereby, induced weight loss, which ameliorates hypertriglyceridemia, hyperglycemia and increases systemic insulin sensitivity.

As also indicated above and set forth in Applicant's priority U.S. application Ser. No. 18/430,796, the PYY secretion induced by the GLP-1/PYY and GLP-1/GIP compositions of the invention promotes satiety and also decreases the rate of gastric emptying, whereby weight loss and, thereby, increased systemic insulin sensitivity are further induced and hypertriglyceridemia and hyperglycemia are further ameliorated.

Thus, in some embodiments of the invention, the GLP-1/PYY, GIP and GLP-1/GIP compositions of the invention are adapted to effectively and safely treat atherosclerosis, i.e., inhibit the formation and progression of the atherosclerosis, and ameliorate at least one physiological risk factor associated with atherosclerosis and/or at least one seminal pathophysiological effect associated with atherosclerosis when delivered to a patient presenting with atherosclerosis, and, if the patient also presents with obesity or type 2 diabetes mellitus, also treat the obesity and type 2 diabetes mellitus, and ameliorate at least one physiological risk factor associated with type 2 diabetes mellitus.

According to the invention, the noted GLP-1/PYY, GIP and GLP-1/GIP compositions preferably comprise at least one of the natural compounds or ligands referenced above that is adapted to bind to and activate at least one olfactory receptor; specifically, olfactory receptor olfactory receptor OR51E1, olfactory receptor OR1A1 or olfactory receptor OR2C1, and at least one free fatty acid receptor; specifically, free fatty acid receptor FFAR1 or FFAR4, and, in some instances, at least one transient receptor; specifically, transient receptor TRPA1.

In some embodiments, the noted GLP-1/PYY, GIP and GLP-1/GIP compositions preferably comprise at least one of the natural compounds or ligands referenced above that is specifically adapted to bind to and activate at least olfactory receptors OR51E1 and free fatty acid receptor FFAR 1 or FFAR4.

According to the invention, the GLP-1/PYY, GIP and GLP-1/GIP compositions of the invention can also comprise at least one natural compound or ligand that is adapted to bind to and activate olfactory receptor family 51 subfamily E member 2 (OR51E2) of endogenous cells, such as, without limitation, β-ionone, propionic acid and acetate, whereby, when the GLP-1/PYY, GIP and GLP-1/GIP compositions are delivered to a patient presenting with atherosclerosis, the GLP-1/PYY, GIP and GLP-1/GIP compositions induce lactate-mediated M2 polarization of macrophages proximate arterial walls in vivo, whereby vascular inflammation is abated and, hence, treatment of atherosclerosis is further facilitated and/or at least one seminal pathophysiological effect associated with atherosclerosis, i.e. vascular inflammation, is further ameliorated.

According to the invention, the GLP-1/PYY, GIP and GLP-1/GIP compositions of the invention can also comprise at least one natural compound or ligand that is adapted to bind to and activate olfactory receptor family 2 subfamily L member 13 (OR2L13) in vivo, such as, without limitation, (−)-carvone and choline.

Recent studies reflect that, when OR2L13 expressed by platelet cells is activated, adenylate cyclase (AC) is recruited and activated, which cleaves ATP to produce cAMP. The produced cAMP then activates cyclic nucleotide gated Ca²⁺ channels and other proteins, such as protein kinase A (PKA). The activated cyclic nucleotide gated Ca²⁺ channels induce an influx of Ca²⁺. The influx of Ca²⁺ along with the produced cAMP further activates membrane channel Anoctamin 7, which induces a Cl⁻ efflux, whereby platelet degranulation is inhibited, and atherosclerotic platelet aggregation is prevented and, hence, further treatment of the atherosclerosis is provided. See Aggarwal, et al., Targeting an Olfactory Receptor Mitigates High Residual Platelet Reactivity and Arterial Thrombosis Through Actin Cytoskeleton Depolymerization, bioRxiv, v. 9 (2024) and Morrell, et al., Platelet Olfactory Receptor Activation Limits Platelet Reactivity and Growth of Aortic Aneurysms, Journal of Clinical Investigation, v. 132.9 (2022).

In some embodiments of the invention, the GLP-1/PYY, GIP and GLP-1/GIP compositions of the invention can also comprise at least one natural compound or ligand that is adapted to bind to and antagonize, i.e. restrict activity of, at least one 5-HT₂-serotonergic receptor (such as 5-HT_{2A}, 5-HT_{2B} and 5-HT_{2C}) in vivo.

Recent studies reflect that, when a 5-HT₂-serotonergic receptor expressed by platelet cells is antagonized, seminal 5-HT₂ receptor-induce cell signaling processes are abated, including inducement of the Gα_{q} signal transduction pathway. The abated inducement of the Gα_{q} signal transduction pathway prevents that dissociation of the Gαq and β-γ subunits of the 5-HT₂ receptor and, hence, the activation of phospholipase C (PLC) activity by the dissociated Gαq subunit, which subsequently prevents the release of diacylglycerol (DAG) and inositol triphosphate (IP3), and, thereby, inhibits protein kinase C (PKC) activity and intracellular Ca²⁺ release. The inhibition of protein kinase C (PKC) activity and intracellular Ca²⁺ abates the release of platelet granules, whereby atherosclerotic platelet aggregation induced thereby is prevented and, hence, further treatment of the atherosclerosis is provided. See Mohammad-Zadeh, et al., Serotonin: A Review, Journal of Veterinary Pharmacology and Therapeutics, vol. 31.3, pp. 187-199 (2008) and Marcinkowska, et al., Exploring the Antiplatelet Activity of Serotonin 5-HT2A Receptor Antagonists Bearing 6-Fluorobenzo [D] Isoxazol-3-Yl) Propyl) Motif—As Potential Therapeutic Agents in the Prevention of Cardiovascular Diseases Biomedicine & Pharmacotherapy, vol. 145, pg. 112424 (2022).

According to the invention, suitable natural compounds or ligands that are adapted to bind to and antagonize at least one 5-HT₂-serotonergic receptor, comprise, without limitation, the following natural antagonists: 3,3′,4′,5,6,7,8-heptamethoxyflavone (HMF), hesperidin and isoliquiritigenin (ISL).

Various exemplar and, hence, non-limiting, embodiments of GLP-1/PYY and GLP-1/GIP compositions of the invention that are adapted to directly treat atherosclerosis, and ameliorate at least one physiological risk factor and/or at least one seminal pathophysiological effect induced by the atherosclerosis, and concurrently (i) treat atherosclerosis, and ameliorate at least one physiological risk factor and/or at least one seminal pathophysiological effect induced by the atherosclerosis and (ii) treat type 2 diabetes mellitus, and ameliorate at least one physiological risk factor and/or at least one seminal pathophysiological effect induced by the type 2 diabetes mellitus, are set forth below.

In one embodiment of the invention, a GLP-1/PYY secretion composition for treating atherosclerosis presented by a patient comprises at least one receptor activating compound adapted to bind to and activate at least one receptor selected from the group comprising olfactory receptor OR51E1, olfactory receptor OR1A1, olfactory receptor OR2C1 and olfactory receptor OR10J5,
the GLP-1/PYY secretion composition adapted to induce GLP-1 and PYY secretion, and, thereby, increased insulin secretion in vivo, whereby the atherosclerosis is treated when the GLP-1/PYY secretion composition is delivered to a patient.

In a preferred embodiment, the GLP-1/PYY secretion composition is further adapted to ameliorate at least one at least one risk factor associated with the atherosclerosis when the GLP-1/PYY secretion composition is delivered to the patient.

In some embodiments, the GLP-1/PYY secretion composition is further adapted to ameliorate at least one seminal pathophysiological effect associated with the atherosclerosis when the GLP-1/PYY secretion composition is delivered to the patient.

In a preferred embodiment, the receptor activating compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In a preferred embodiment, the receptor activating compound is adapted to induce at least 50% activation of at least olfactory receptor OR51E1 in vivo when the GLP-1/PYY secretion composition is delivered to the patient.

In another embodiment of the invention, a GLP-1/PYY secretion composition for treating atherosclerosis presented by a patient comprises at least one receptor activating compound adapted to bind to and activate at least one receptor selected from the group comprising olfactory receptor OR51E1 and olfactory receptor OR1A1,
the GLP-1/PYY secretion composition adapted to induce GLP-1 and PYY secretion, and, thereby, increased insulin secretion in vivo, whereby the atherosclerosis is treated when the GLP-1/PYY secretion composition is delivered to a patient.

In a preferred embodiment, the GLP-1/PYY secretion composition is further adapted to ameliorate at least one at least one risk factor associated with the atherosclerosis when the GLP-1/PYY secretion composition is delivered to the patient.

In some embodiments, the GLP-1/PYY secretion composition is further adapted to ameliorate at least one seminal pathophysiological effect associated with the atherosclerosis when the GLP-1/PYY secretion composition is delivered to the patient.

In some embodiments, the receptor activating compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the receptor activating compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 30.0 μM.

In a preferred embodiment, the receptor activating compound is adapted to induce at least 50% activation of at least olfactory receptor OR51E1 in vivo when the GLP-1/PYY secretion composition is delivered to the patient.

In another embodiment of the invention, a GLP-1/PYY secretion composition for treating atherosclerosis presented by a patient comprises a plurality of receptor activating compounds adapted to bind to and activate at least olfactory receptor OR51E1 and olfactory receptor OR1A1,
the GLP-1/PYY secretion composition adapted to induce GLP-1 and PYY secretion, and, thereby, increased insulin secretion in vivo, whereby the atherosclerosis is treated when the GLP-1/PYY secretion composition is delivered to a patient.

In a preferred embodiment, the GLP-1/PYY secretion composition is further adapted to ameliorate at least one at least one risk factor associated with the atherosclerosis when the GLP-1/PYY secretion composition is delivered to the patient.

In some embodiments, the GLP-1/PYY secretion composition is further adapted to ameliorate at least one seminal pathophysiological effect associated with the atherosclerosis when the GLP-1/PYY secretion composition is delivered to the patient.

In a preferred embodiment, the plurality of receptor activating compounds comprise eugenol, 3-methylpentanoic acid and geraniol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/PYY secretion composition in the range of approximately 10.0 μM to approximately 30.0 μM.

In some embodiments, the geraniol comprises an EC₅₀ value of at least approximately 0.1 μM in the GLP-1/PYY secretion composition.

In some embodiments, the geraniol comprises an EC₅₀ value in the GLP-1/GIP secretion composition in the range of approximately 1.0 μM to approximately 15.0 μM.

In a preferred embodiment, at least one of the plurality of receptor activating compounds is adapted to induce at least 50% activation of at least olfactory receptor OR51E1 in vivo when the GLP-1/PYY secretion composition is delivered to the patient.

In another embodiment of the invention, a GLP-1/PYY secretion composition for treating a plurality of physiological disorders presenting by a patient comprises a plurality of receptor activating compounds adapted to bind to and activate at least olfactory receptor OR51E1 and olfactory receptor OR1A1, the GLP-1/PYY secretion composition similarly adapted to induce GLP-1 and PYY secretion, and, thereby, increased insulin secretion in vivo, whereby at least one of the plurality of physiological disorders is treated when the GLP-1/PYY secretion composition is delivered to a patient.

In a preferred embodiment, the plurality of physiological disorders comprises atherosclerosis and an insulin-resistance-induced physiological disorder.

In some embodiments, the insulin-resistance-induced physiological disorder comprises type 2 diabetes mellitus.

In a preferred embodiment, the plurality of receptor activating compounds comprise eugenol and geraniol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the geraniol comprises an EC₅₀ value of at least approximately 0.1 μM in the GLP-1/PYY secretion composition.

In some embodiments, the geraniol comprises an EC₅₀ value in the GLP-1/GIP secretion composition in the range of approximately 1.0 μM to approximately 15.0 μM.

In a preferred embodiment, at least one of the receptor activating compounds is adapted to induce at least 50% activation of at least OR51E1 in vivo when the GLP-1/PYY secretion composition is delivered to the patient.

In another embodiment of the invention, a GLP-1/GIP secretion composition for treating atherosclerosis presented by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5, and
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1,
the GLP-1/GIP secretion composition adapted to induce GLP-1 and GIP secretion, and, thereby, increased insulin secretion in vivo, whereby the atherosclerosis is treated when the GLP-1/GIP secretion composition is delivered to a patient.

In a preferred embodiment, the GLP-1/GIP secretion composition is further adapted to ameliorate at least one risk factor associated with the atherosclerosis when the GLP-1/GIP secretion composition is delivered to the patient.

In some embodiments, the GLP-1/GIP secretion composition is further adapted to ameliorate at least one seminal pathophysiological effect associated with the atherosclerosis when the GLP-1/GIP secretion composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/GIP secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In some embodiments, the cinnamaldehyde comprises an EC₅₀ value in the GLP-1/GIP secretion composition in the range of approximately 0.1 μM to approximately 2500.0 μM.

In a preferred embodiment, at least one of the first receptor activating compounds is adapted to induce at least 50% activation of at least olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In another embodiment of the invention, a GLP-1/GIP secretion composition for treating a plurality of physiological disorders presenting by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5, and
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1,
the GLP-1/GIP secretion composition adapted to induce GLP-1 and GIP secretion, and, thereby, increased insulin secretion in vivo, whereby at least one of the plurality of physiological disorders is treated when the GLP-1/PYY secretion composition is delivered to a patient.

In a preferred embodiment, the plurality of physiological disorders comprises atherosclerosis and an insulin-resistance-induced physiological disorder.

In some embodiments, the insulin-resistance-induced physiological disorder comprises type 2 diabetes mellitus.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 50.0 μM.

In a preferred embodiment, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/GIP secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In some embodiments, the cinnamaldehyde comprises an EC₅₀ value in the GLP-1/GIP secretion composition in the range of approximately 0.1 μM to approximately 2500.0 μM.

In a preferred embodiment, at least one of the first receptor activating compounds is adapted to induce at least 50% activation of at least olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In another embodiment of the invention, a GLP-1/GIP secretion composition for treating atherosclerosis presented by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5,
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1, and
(iii) at least a third receptor activating compound adapted to bind to and activate olfactory receptor OR51E2,
the GLP-1/GIP secretion composition adapted to induce GLP-1 and GIP secretion, and, thereby, increased insulin secretion in vivo, whereby the atherosclerosis is treated when the GLP-1/GIP secretion composition is delivered to a patient.

In a preferred embodiment, the GLP-1/GIP secretion composition is further adapted to ameliorate at least one risk factor associated with the atherosclerosis when the GLP-1/GIP secretion composition is delivered to the patient.

In some embodiments, the GLP-1/GIP secretion composition is further adapted to ameliorate at least one seminal pathophysiological effect associated with the atherosclerosis when the GLP-1/GIP secretion composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/GIP secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In some embodiments, the cinnamaldehyde comprises an EC₅₀ value in the GLP-1/GIP secretion composition in the range of approximately 0.1 μM to approximately 2500.0 μM.

In some embodiments, the third receptor activating compound comprises a fourth compound selected from the group comprising β-ionone, propionic acid and acetate.

In some embodiments, the fourth compound comprises β-ionone.

In some embodiments, the β-ionone comprises an EC₅₀ value of at least approximately 0.1 μM in the GLP-1/GIP secretion composition.

In some embodiments, the β-ionone comprises an EC₅₀ value in the GLP-1/GIP secretion composition in the range of approximately 5.0 μM to approximately 250.0 μM.

In some embodiments, the fourth compound comprises propionic acid.

In some embodiments, the propionic acid comprises an EC₅₀ value of at least approximately 0.05 μM in the GLP-1/GIP secretion composition.

In some embodiments, the propionic acid comprises an EC₅₀ value in the GLP-1/GIP secretion composition in the range of approximately 0.05 μM to approximately 0.5 μM.

In a preferred embodiment, at least one of the first receptor activating compounds is adapted to induce at least 50% activation of at least olfactory receptor olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In a preferred embodiment, at least one of the third receptor activating compounds is adapted to induce at least 50% activation of olfactory receptor OR51E2 in vivo when the GLP-1/GIP secretion composition is delivered to the patient.

In another embodiment of the invention, a GLP-1/GIP secretion composition for treating atherosclerosis presented by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5,
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1, and
(iii) at least a second receptor activating compound adapted to bind to and activate olfactory receptor OR2L13,
the GLP-1/GIP secretion composition adapted to induce GLP-1 and GIP secretion, and, thereby, increased insulin secretion in vivo, whereby the atherosclerosis is treated when the GLP-1/GIP secretion composition is delivered to a patient.

In a preferred embodiment, the GLP-1/GIP secretion composition is further adapted to ameliorate at least one risk factor associated with the atherosclerosis when the GLP-1/GIP secretion composition is delivered to the patient.

In some embodiments, the GLP-1/GIP secretion composition is further adapted to ameliorate at least one seminal pathophysiological effect associated with the atherosclerosis when the GLP-1/GIP secretion composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/GIP secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In some embodiments, the cinnamaldehyde comprises an EC₅₀ value in the GLP-1/GIP secretion composition in the range of approximately 0.1 μM to approximately 2500.0 μM.

In a preferred embodiment, the third receptor activating compound comprises a third compound selected from the group comprising (−)-carvone and choline.

In some embodiments, the third receptor activating compound comprises (−)-carvone.

In some embodiments, the (−)-carvone comprises an EC₅₀ value of at least approximately 0.1 μM in the GLP-1/GIP secretion composition.

In some embodiments, the (−)-carvone comprises an EC₅₀ value in the GLP-1/GIP secretion composition in the range of approximately 0.01 μM to approximately 100.0 μM.

In a preferred embodiment, at least one of the first receptor activating compounds is adapted to induce at least 50% activation of at least olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In a preferred embodiment, the third receptor activating compound is adapted to induce at least 50% activation of olfactory receptor OR2L13 in vivo when the GLP-1/GIP secretion composition is delivered to the patient.

In another embodiment of the invention, a GLP-1/GIP secretion composition for treating atherosclerosis presented by a patient comprises:
(i) at least a first receptor activating compound adapted to bind to and activate at least one first receptor selected from the group comprising olfactory receptor OR51E1, olfactory receptor OR1A1 and olfactory receptor OR2C1,
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1, and
(iii) at least a third receptor activating compound adapted to bind to and antagonize at least a 5-HT₂-serotongeric receptor,
the GLP-1/GIP secretion composition adapted to induce GLP-1 secretion, and, thereby, increased insulin secretion, and restrict 5-HT₂-serotongeric receptor activity in vivo, whereby the atherosclerosis is treated when the GLP-1/GIP secretion composition is delivered to a patient.

In a preferred embodiment, the GLP-1/GIP secretion composition is further adapted to ameliorate at least one risk factor associated with the atherosclerosis when the GLP-1/GIP secretion composition is delivered to the patient.

In some embodiments, the GLP-1/GIP secretion composition is further adapted to ameliorate at least one seminal pathophysiological effect associated with the atherosclerosis when the GLP-1/GIP secretion composition is delivered to the patient.

In some embodiments, the first receptor activating compound comprises a first compound selected from the group comprising eugenol, 3-methylpentanoic acid and geraniol.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/GIP secretion composition.

In some embodiments, the lauric acid comprises an EC₅₀ value in the GLP-1/GIP secretion composition in the range of approximately 0.05 μM to approximately 50.0 μM.

In some embodiments, the second compound comprises a long-chain free fatty acid.

In some embodiments, the long-chain free fatty acid comprises stearic acid.

In some embodiments, the stearic acid comprises an EC₅₀ value of at least approximately 0.05 μM in the GLP-1/GIP secretion composition.

In some embodiments, the stearic acid comprises an EC₅₀ value in the GLP-1/GIP secretion composition in the range of approximately 0.05 μM to approximately 0.5 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In some embodiments, the cinnamaldehyde comprises an EC₅₀ value in the GLP-1/GIP secretion composition in the range of approximately 0.1 μM to approximately 2500.0 μM.

In a preferred embodiment, the third receptor activating compound comprises a fourth compound selected from the group comprising 3,3′,4′,5,6,7,8-heptamethoxyflavone (HMF), hesperidin and isoliquiritigenin (ISL).

In some embodiments, the third receptor activating compound comprises HMF.

In some embodiments, the HMF comprises an EC₅₀ value of at least approximately 0.01 μM in the GLP-1/GIP secretion composition.

In a preferred embodiment, at least one of the first receptor activating compounds is adapted to induce at least 50% activation of at least olfactory receptor OR51E1 in vivo when the GLP-1/GIP secretion composition is delivered to the patient.

### Diabetic Cardiomyopathy

Diabetic cardiomyopathy (DCM) is a cardiovascular disease that is characterized by the development of abnormal myocardial structure and performance in the absence of typical cardiovascular disease physiological risk factors, such as coronary artery disease, hypertension, and valvular disease, in individuals afflicted with diabetes mellitus. DCM induces seminal pathophysiological changes in the myocardial structure, including abnormal thickening of the myocardium; particularly, the myocardium of the left ventricle (LV) due to LV hypertrophy, myocardial fibrosis, and maladaptive changes to myocardial cell signaling.

The abnormal thickening of the LV can and, often does, reduce the space within the LV chamber, thereby impairing ventricular filling and reducing blood flow from the heart into the aorta. The noted pathophysiological changes in the myocardial structure and associated subclinical diastolic dysfunction often lead to heart failure with normal ejection fraction and eventually progresses to systolic dysfunction accompanied by heart failure with reduced ejection fraction.

There are two main forms of DCM: (i) DCM with preserved ejection fraction and (ii) DCM with reduced ejection fraction.

As discussed in detail below, DCM with preserved ejection fraction occurs when the abnormally thickened LV fails to relax properly, which prevents the LV chamber from filling completely resulting in diastolic dysfunction. Ejection fraction is, however, normal or near normal (i.e., at least between 50-70%) despite the diastolic dysfunction.

DCM with reduced ejection fraction similarly occurs when the abnormally thickened myocardium fails to relax properly, which prevents the LV chamber from filling completely resulting in diastolic dysfunction. In this instance, however, the ejection fraction is abnormally low (i.e., 49% and below) and attendant systolic dysfunction is present.

As discussed in detail below, in some embodiments of the invention, the GLP-1/PYY, GIP and GLP-1/GIP compositions of the invention are adapted to effectively and safely (i) treat DCM, (ii) ameliorate and/or stabilize at least one physiological risk factor associated with DCM, and (iii) preferably, ameliorate and/or stabilize at least one pathophysiological effect induced by DCM, when the GLP-1/PYY, GIP and GLP-1/GIP compositions are delivered to a patient presenting with DCM.

Indeed, according to the invention, in some embodiments, when the GLP-1/PYY, GIP and GLP-1/GIP compositions of the invention are delivered to a patient presenting with DCM, the GLP-1/PYY, GIP and GLP-1/GIP compositions effectively and safely treat the DCM, i.e., inhibit the formation and progression of DCM, and ameliorate at least one physiological risk factor associated with DCM and/or at least one seminal pathophysiological effect associated with DCM by (i) increasing autophagy of damaged cardiomyocytes, (ii) decreasing pro-inflammatory cytokine activity and (iii) decreasing attendant pro-inflammatory ROS generation by restoring the expression of catalase and manganese superoxide dismutase (MnSOD).

In some embodiments of the invention, the GLP-1/PYY, GIP and GLP-1/GIP secretion compositions also delay the progression of DCM, and ameliorate at least one physiological risk factor of DCM and/or at least one seminal pathophysiological effect associated with DCM by decreasing insulin resistance-induced apoptosis of cardiomyocytes resulting from hyperglycemia associated glucotoxicity via the RAGE receptor, decreasing apoptosis of vascular endothelial and smooth muscle cells and reversing maladaptive vascular remodeling (including vascular fibrosis and intimal neoplasia) and luminal stenosis.

In some embodiments, the GLP-1/PYY, GIP and GLP-1/GIP secretion compositions also (i) induce OR10J5-dependent phosphorylation of Akt kinase and, thereby, angiogenesis and blood vessel formation in the myocardium, which promotes remodeling of damaged myocardium without fibrosis of the myocardium, and (ii) activates and, hence, modulates OR51E1 of myocardial cells, which exerts a negative inotropic effect and a negative chronotropic effect and, thereby, reduced myocardial stress on the heart.

Thus, according to the invention, in some embodiments, the GLP-1/PYY, GIP and GLP-1/GIP compositions of the invention are adapted to effectively and safely treat DCM presented by a patient, i.e., inhibit the formation and progression of DCM, by inducing multiple seminal physiological processes, including, without limitation, (i) increasing systemic insulin sensitivity, (ii) increasing autophagy of damaged cardiomyocytes, (iii) decreasing pro-inflammatory cytokine activity and (iv) decreasing attendant pro-inflammatory ROS generation by restoring the expression of catalase and manganese superoxide dismutase (MnSOD).

As indicated above, according to the invention, the noted GLP-1/PYY, GIP and GLP-1/GIP compositions are also adapted to ameliorate at least one physiological risk factor associated with DCM, including, without limitation, systemic insulin resistance, impaired insulin production, and insulin resistance-induced apoptosis of cardiomyocytes.

As also indicated above, the noted GLP-1/PYY, GIP and GLP-1/GIP compositions of the invention are also preferably adapted to ameliorate at least one pathophysiological effect associated with DCM, including, without limitation, endothelial dysfunction, e.g., apoptosis of vascular endothelial and smooth muscle cells, maladaptive vascular remodeling (including vascular fibrosis and intimal neoplasia) and luminal stenosis.

Recent studies reflect that there is similarly a strong correlation between the physiological risk factors associated with insulin-resistance-induced physiological disorders; particularly, type 2 diabetes mellitus, and, hence, the physiological risk factors associated therewith; particularly, hyperglycemia, and DCM. See Jia, et al., Diabetic Cardiomyopathy: A Hyperglycemia and Insulin-Resistance Induced Heart Disease, Diabetologia, vol. 61.1, pp. 21-28 (2018) and Austin, et al., Hypertriglyceridemia as a Cardiovascular Risk, American Journal of Cardiology, v. 81(4), pp. 7B-12B (1998).

Indeed, the studies reflect that insulin resistance and, hence, hyperglycemia associated glucotoxicity resulting therefrom, induces a protein glycation reaction resulting in increased production of advanced glycation end products (AGEs), which are produced from non-enzymatic glycosylation of lipids, lipoproteins and amino acids. Increased AGE production induces maladaptive myocardial remodeling and, hence, changes in mechanical properties of the myocardial extracellular matrix (ECM), e.g., increased myocardial ECM stiffness and impaired diastolic relaxation, by increasing resistance of the myocardial ECM to connective tissue enzymatic proteolysis and inducing the fibrotic crosslinking of the myocardial ECM.

The AGEs also bind to the cardiomyocyte cell surface receptor for AGE (RAGE) to (i) induce maladaptive inflammatory gene expression, (ii) increase myocardial ECM protein production via processes mediated via through mitogen-activated protein kinase (MAPK) and Janus kinase (JAK) pathways in the myocardium and (iii) increase the production of pro-inflammatory reactive oxygen species (ROS), which promote further fibrotic crosslinking of the myocardial ECM.

In some instances, the binding of AGEs to the RAGE receptor of cardiomyocytes also induces apoptosis of damaged cardiomyocytes and, hence, a pro-inflammatory reaction to the apoptosis of the cardiomyocytes and the pro-apoptotic (and pro-inflammatory) cytokines released thereby that promotes further fibrotic crosslinking of the myocardial ECM.

The relationship between insulin resistance and DCM is further reflected by insulin resistance of the myocardium of the heart, i.e., a seminal marker of DCM.

Under normal physiological conditions, the PI3K/Akt signaling pathway in the cells of the myocardium, e.g., cardiomyocytes, stimulates GLUT4 recruitment to the plasma membranes thereof, which results in glucose uptake into the cells of the myocardium.

When a patient presents with DCM and, hence, systemic insulin resistance, the PI3K/Akt signaling pathway is impaired, which results in a reduction of systemic glucose uptake and, thereby decreases Ca²⁺ ATPase activity and transports the Ca²⁺ back into the sarcoplasmic reticulum, thus, increasing intracellular Ca²⁺.

The insulin resistance further inhibits cardiac insulin-stimulated coronary endothelial NO synthase activity and NO production, which further increases intracellular Ca²⁺ levels and Ca²⁺ sensitization in the cells via the cGMP/PKG signaling pathway. The maladaptive changes to cell signaling result in seminal pathophysiological effects, including endoplasmic reticular (ER) stress and impaired Ca²⁺ homeostasis, which result in the apoptosis of the cells and pro-inflammatory responses thereto that are associated with maladaptive myocardial remodeling (e.g., myocardial fibrosis and, hence, stiffness and impaired diastolic relaxation) and attendant diastolic dysfunction.

There is, thus, similarly a strong correlation between the physiological risk factors associated with insulin-resistance-induced physiological disorders; particularly, type 2 diabetes mellitus, and DCM.

As indicated above, there is also a strong correlation between obesity and type 2 diabetes mellitus. See, e.g., Guilherme, et al., Adipocyte Dysfunctions Linking Obesity to Insulin Resistance and Type 2 Diabetes, supra.

As indicated above, in some embodiments of the invention, when the GLP-1/PYY, GIP and GLP-1/GIP compositions of the invention are delivered to a patient presenting with DCM, the GLP-1/PYY, GIP and GLP-1/GIP compositions of the invention will effectively and safely treat the DCM, and ameliorate and/or stabilize at least one physiological risk factor associated with DCM and at least one pathophysiological effect associated with DCM.

In a preferred embodiment, when the patient also presents with insulin resistance of the myocardium, the GLP-1/PYY, GIP and GLP-1/GIP compositions of the will also effectively and safely treat the insulin resistance of the myocardium.

As indicated above and set forth in Applicant's priority U.S. application Ser. No. 18/980,129, when the GLP-1/PYY, GIP and GLP-1/GIP compositions of the invention are delivered to a patient presenting with obesity and/or type 2 diabetes mellitus, the GLP-1/PYY, GIP and GLP-1/GIP compositions of the invention will also effectively and safely treat the obesity and/or treat type 2 diabetes mellitus, and ameliorate at least one physiological risk factor associated with type 2 diabetes mellitus.

Thus, in some embodiments of the invention, the GLP-1/PYY, GIP and GLP-1/GIP compositions of the invention are preferably adapted to effectively and safely treat DCM, and ameliorate at least one physiological risk factor associated with DCM and/or at least one seminal pathophysiological effect associated with DCM when delivered to a patient presenting with DCM, and, if the patient also presents with obesity or type 2 diabetes mellitus or insulin resistance of the myocardium, also treat the obesity, treat the type 2 diabetes mellitus, and ameliorate at least one physiological risk factor associated with type 2 diabetes mellitus, and treat the insulin resistance of the myocardium.

According to the invention, the noted GLP-1/PYY, GIP and GLP-1/GIP compositions similarly preferably comprise at least one of the natural compounds or ligands referenced above that is adapted to bind to and activate at least one olfactory receptor; specifically, olfactory receptor OR51E1, olfactory receptor OR1A1 or olfactory receptor OR2C1, and at least one free fatty acid receptor; specifically, free fatty acid receptor FFAR1 or free fatty acid receptor FFAR4, and, in some instances, at least one transient receptor; specifically, transient receptor TRPA1.

In some embodiments, the noted GLP-1/PYY, GIP and GLP-1/GIP compositions similarly preferably comprise at least one of the natural compounds or ligands referenced above that is specifically adapted to bind to and activate at least olfactory receptor OR51E1 and free fatty acid receptor FFAR 1 or free fatty acid receptor FFAR4.

According to the invention, the noted GLP-1/PYY, GIP and GLP-1/GIP compositions of the invention can also similarly comprise at least one natural compound or ligand that is adapted to bind to and activate olfactory receptor OR51E2 of endogenous cells, such as, without limitation, β-ionone, propionic acid and acetate, whereby, when the GLP-1/PYY, GIP and GLP-1/GIP compositions are delivered to a patient presenting with DCM, the GLP-1/PYY, GIP and GLP-1/GIP compositions inhibit secretion of pro-inflammatory cytokines, including TNF-α and IL-6 in vivo, whereby myocardium inflammation is abated and, hence, treatment of DCM is further facilitated and/or at least one seminal pathophysiological effect associated with DCM is further ameliorated.

Various exemplar and, hence, non-limiting, embodiments of GLP-1/PYY and GLP-1/GIP compositions of the invention that are adapted to directly treat DCM, and ameliorate at least one physiological risk factor of DCM and/or at least one seminal pathophysiological effect induced by DCM, and concurrently (i) treat DCM, and ameliorate at least one physiological risk factor of DCM and/or at least one seminal pathophysiological effect induced by DCM, and (ii) treat type 2 diabetes mellitus, and ameliorate at least one physiological risk factor of type 2 diabetes mellitus and at least one seminal pathophysiological effect induced by type 2 diabetes mellitus and (iii) treat insulin resistance of the myocardium, are set forth below.

In one embodiment of the invention, a GLP-1/PYY secretion composition for treating diabetic cardiomyopathy presented by a patient comprises at least one receptor activating compound adapted to bind to and activate at least one receptor selected from the group comprising olfactory receptor OR51E1, olfactory receptor OR1A1, olfactory receptor OR2C1 and olfactory receptor OR10J5,
the GLP-1/PYY secretion composition adapted to induce GLP-1 and PYY secretion, and, thereby, increased insulin secretion in vivo, whereby the diabetic cardiomyopathy is treated when the GLP-1/PYY secretion composition is delivered to a patient.

In a preferred embodiment, the GLP-1/PYY secretion composition is further adapted to ameliorate at least one at least one risk factor associated with the diabetic cardiomyopathy when the GLP-1/PYY secretion composition is delivered to the patient.

In some embodiments, the GLP-1/PYY secretion composition is further adapted to ameliorate at least one seminal pathophysiological effect associated with the diabetic cardiomyopathy when the GLP-1/PYY secretion composition is delivered to the patient.

In some embodiments, the receptor activating compound comprises eugenol.

In some embodiments, the eugenol comprises an EC₅₀ value of at least approximately 0.1 μM in the GLP-1/PYY secretion composition.

In some embodiments, the receptor activating compound comprises 3-methylpentanoic acid.

In some embodiments, the 3-methylpentanoic acid comprises an EC₅₀ value of at least approximately 0.1 μM in the GLP-1/PYY secretion composition.

In a preferred embodiment, the receptor activating compound is adapted to induce at least 50% activation of at least olfactory receptor OR51E1 in vivo when the GLP-1/PYY secretion composition is delivered to the patient.

In another embodiment of the invention, a GLP-1/PYY secretion composition for treating diabetic cardiomyopathy presented by a patient comprises at least one receptor activating compound adapted to bind to and activate at least one receptor selected from the group comprising olfactory receptor OR51E1 and olfactory receptor OR1A1,
the GLP-1/PYY secretion composition adapted to induce GLP-1 and PYY secretion, and, thereby, increased insulin secretion in vivo, whereby the diabetic cardiomyopathy is treated when the GLP-1/PYY secretion composition is delivered to a patient.

In a preferred embodiment, the GLP-1/PYY secretion composition is further adapted to ameliorate at least one at least one risk factor associated with the diabetic cardiomyopathy when the GLP-1/PYY secretion composition is delivered to the patient.

In some embodiments, the GLP-1/PYY secretion composition is further adapted to ameliorate at least one seminal pathophysiological effect associated with the diabetic cardiomyopathy when the GLP-1/PYY secretion composition is delivered to the patient.

In some embodiments, the receptor activating compound comprises eugenol.

In some embodiments, the eugenol comprises an EC₅₀ value in the GLP-1/GIP secretion composition in the range of approximately 1.0 μM to approximately 15.0 μM.

In some embodiments, the receptor activating compound comprises 3-methylpentanoic acid.

In some embodiments, the 3-methylpentanoic acid comprises an EC₅₀ value in the GLP-1/GIP secretion composition in the range of approximately 10.0 μM to approximately 30.0 μM.

In a preferred embodiment, the receptor activating compound is adapted to induce at least 50% activation of at least olfactory receptor OR51E1 in vivo when the GLP-1/PYY secretion composition is delivered to the patient.

In another embodiment of the invention, a GLP-1/PYY secretion composition for treating diabetic cardiomyopathy presented by a patient comprises a plurality of receptor activating compounds adapted to bind to and activate at least olfactory receptor OR51E1 and olfactory receptor OR1A1,
the GLP-1/PYY secretion composition adapted to induce GLP-1 and PYY secretion, and, thereby, increased insulin secretion in vivo, whereby the diabetic cardiomyopathy is treated when the GLP-1/PYY secretion composition is delivered to a patient.

In a preferred embodiment, the GLP-1/PYY secretion composition is further adapted to ameliorate at least one at least one risk factor associated with the diabetic cardiomyopathy when the GLP-1/PYY secretion composition is delivered to the patient.

In some embodiments, the GLP-1/PYY secretion composition is further adapted to ameliorate at least one seminal pathophysiological effect associated with the diabetic cardiomyopathy when the GLP-1/PYY secretion composition is delivered to the patient.

In some embodiments, the plurality of receptor activating compounds comprise eugenol and geraniol.

In some embodiments, the eugenol comprises an EC₅₀ value in the GLP-1/GIP secretion composition in the range of approximately 1.0 μM to approximately 30.0 μM.

In some embodiments, the geraniol comprises an EC₅₀ value in the GLP-1/GIP secretion composition in the range of approximately 1.0 μM to approximately 30.0 μM.

In a preferred embodiment, at least one of the plurality of receptor activating compounds is adapted to induce at least 50% activation of at least olfactory receptor OR51E1 in vivo when the GLP-1/PYY secretion composition is delivered to the patient.

In another embodiment of the invention, a GLP-1/PYY secretion composition for treating a plurality of physiological disorders presenting by a patient comprises a plurality of receptor activating compounds adapted to bind to and activate at least olfactory receptor OR51E1 and olfactory receptor OR1A1,
the GLP-1/PYY secretion composition similarly adapted to induce GLP-1 and PYY secretion, and, thereby, increased insulin secretion in vivo, whereby at least one of the plurality of physiological disorders is treated when the GLP-1/PYY secretion composition is delivered to a patient.

In a preferred embodiment, the plurality of physiological disorders comprises diabetic cardiomyopathy and an insulin-resistance-induced physiological disorder.

In some embodiments, the insulin-resistance-induced physiological disorder comprises type 2 diabetes mellitus.

In a preferred embodiment, the plurality of receptor activating compounds comprise eugenol and geraniol.

In some embodiments, the plurality of receptor activating compounds comprise eugenol and geraniol.

In some embodiments, the eugenol comprises an EC₅₀ value in the GLP-1/GIP secretion composition in the range of approximately 1.0 μM to approximately 30.0 μM.

In some embodiments, the geraniol comprises an EC₅₀ value in the GLP-1/GIP secretion composition in the range of approximately 1.0 μM to approximately 30.0 μM.

In a preferred embodiment, at least one of the receptor activating compounds is adapted to induce at least 50% activation of at least OR51E1 in vivo when the GLP-1/PYY secretion composition is delivered to the patient.

In another embodiment of the invention, a GLP-1/GIP secretion composition for treating diabetic cardiomyopathy presented by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5, and
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1,
the GLP-1/GIP secretion composition adapted to induce GLP-1 and GIP secretion, and, thereby, increased insulin secretion in vivo, whereby the diabetic cardiomyopathy is treated when the GLP-1/GIP secretion composition is delivered to a patient.

In a preferred embodiment, the GLP-1/GIP secretion composition is further adapted to ameliorate at least one risk factor associated with the diabetic cardiomyopathy when the GLP-1/GIP secretion composition is delivered to the patient.

In some embodiments, the GLP-1/GIP secretion composition is further adapted to ameliorate at least one seminal pathophysiological effect associated with the diabetic cardiomyopathy when the GLP-1/GIP secretion composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value in the GLP-1/GIP secretion composition in the range of approximately 1.0 μM to approximately 15.0 μM.

In some embodiments, the first compound comprises 3-methylpentanoic acid.

In some embodiments, the 3-methylpentanoic acid comprises an EC₅₀ value in the GLP-1/GIP secretion composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value in the GLP-1/GIP secretion composition in the range of approximately 0.05 μM to approximately 50.0 μM.

In some embodiments, the second receptor activating compound comprises cinnamaldehyde.

In some embodiments, the cinnamaldehyde comprises an EC₅₀ value in the GLP-1/GIP secretion composition in the range of approximately 0.1 μM to approximately 2500.0 μM.

In a preferred embodiment, at least one of the first receptor activating compounds is adapted to induce at least 50% activation of at least olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In another embodiment of the invention, a GLP-1/GIP secretion composition for treating a plurality of physiological disorders presenting by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5, and
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1,
the GLP-1/GIP secretion composition adapted to induce GLP-1 and GIP secretion, and, thereby, increased insulin secretion in vivo, whereby at least one of the plurality of physiological disorders is treated when the GLP-1/PYY secretion composition is delivered to a patient.

In a preferred embodiment, the plurality of physiological disorders comprises diabetic cardiomyopathy and an insulin-resistance-induced physiological disorder.

In a preferred embodiment, the GLP-1/GIP secretion composition is further adapted to ameliorate at least one risk factor associated with the diabetic cardiomyopathy when the GLP-1/GIP secretion composition is delivered to the patient.

In some embodiments, the GLP-1/GIP secretion composition is further adapted to ameliorate at least one seminal pathophysiological effect associated with the diabetic cardiomyopathy when the GLP-1/GIP secretion composition is delivered to the patient.

In some embodiments, the insulin-resistance-induced physiological disorder comprises type 2 diabetes mellitus.

In some embodiments, the GLP-1/GIP secretion composition is further adapted to ameliorate at least one risk factor associated with the type 2 diabetes mellitus when the GLP-1/GIP secretion composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the GLP-1/PYY secretion composition.

In some embodiments, the first compound comprises 3-methylpentanoic acid.

In some embodiments, the 3-methylpentanoic acid comprises an EC₅₀ value of at least approximately 0.1 μM in the GLP-1/PYY secretion composition.

In a preferred embodiment, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/GIP secretion composition.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In a preferred embodiment, at least one of the first receptor activating compounds is adapted to induce at least 50% activation of at least olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In another embodiment of the invention, a GLP-1/GIP secretion composition for treating diabetic cardiomyopathy presented by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5,
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1, and
(iii) at least a third receptor activating compound adapted to bind to and activate olfactory receptor OR51E2,
the GLP-1/GIP secretion composition adapted to induce GLP-1 and GIP secretion, and, thereby, increased insulin secretion in vivo, whereby the diabetic cardiomyopathy is treated when the GLP-1/GIP secretion composition is delivered to a patient.

In a preferred embodiment, the GLP-1/GIP secretion composition is further adapted to ameliorate at least one risk factor associated with the diabetic cardiomyopathy when the GLP-1/GIP secretion composition is delivered to the patient.

In some embodiments, the GLP-1/GIP secretion composition is further adapted to ameliorate at least one seminal pathophysiological effect associated with the diabetic cardiomyopathy when the GLP-1/GIP secretion composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value in the GLP-1/GIP secretion composition in the range of approximately 1.0 μM to approximately 30.0 μM.

In some embodiments, the first compound comprises 3-methylpentanoic acid.

In some embodiments, the 3-methylpentanoic acid comprises an EC₅₀ value in the GLP-1/GIP secretion composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value in the GLP-1/GIP secretion composition in the range of approximately 0.1 μM to approximately 2500.0 μM.

In some embodiments, the third receptor activating compound comprises a fourth compound selected from the group comprising β-ionone, propionic acid and acetate.

In some embodiments, the fourth compound comprises β-ionone.

In some embodiments, the β-ionone comprises an EC₅₀ value of at least approximately 0.1 μM in the GLP-1/GIP secretion composition.

In a preferred embodiment, at least one of the first receptor activating compounds is adapted to induce at least 50% activation of at least olfactory receptor olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In a preferred embodiment, at least one of the third receptor activating compounds is adapted to induce at least 50% activation of olfactory receptor OR51E2 in vivo when the GLP-1/GIP secretion composition is delivered to the patient.

In another embodiment of the invention, a GLP-1/GIP secretion composition for treating diabetic cardiomyopathy presented by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5,
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1, and
(iii) at least a second receptor activating compound adapted to bind to and activate olfactory receptor OR2L13,
the GLP-1/GIP secretion composition adapted to induce GLP-1 and GIP secretion, and, thereby, increased insulin secretion in vivo, whereby the diabetic cardiomyopathy is treated when the GLP-1/GIP secretion composition is delivered to a patient.

In a preferred embodiment, the GLP-1/GIP secretion composition is further adapted to ameliorate at least one risk factor associated with the diabetic cardiomyopathy when the GLP-1/GIP secretion composition is delivered to the patient.

In some embodiments, the GLP-1/GIP secretion composition is further adapted to ameliorate at least one seminal pathophysiological effect associated with the diabetic cardiomyopathy when the GLP-1/GIP secretion composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value in the GLP-1/GIP secretion composition in the range of approximately 1.0 μM to approximately 30.0 μM.

In some embodiments, the first compound comprises 3-methylpentanoic acid.

In some embodiments, the 3-methylpentanoic acid comprises an EC₅₀ value in the GLP-1/GIP secretion composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value in the GLP-1/GIP secretion composition in the range of approximately 0.1 μM to approximately 2500.0 μM.

In a preferred embodiment, the third receptor activating compound comprises a third compound selected from the group comprising (−)-carvone and choline.

In some embodiments, the third receptor activating compound comprises (−)-carvone.

In some embodiments, the (−)-carvone comprises an EC₅₀ value of at least approximately 0.1 μM in the GLP-1/GIP secretion composition.

In a preferred embodiment, at least one of the first receptor activating compounds is adapted to induce at least 50% activation of at least olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In a preferred embodiment, the third receptor activating compound is adapted to induce at least 50% activation of olfactory receptor OR2L13 in vivo when the GLP-1/GIP secretion composition is delivered to the patient.

### Hypertension

Hypertension is a chronic medical condition characterized by persistently elevated blood pressure in the arteries. One of the most common types of hypertension is essential (or primary) hypertension, which is characterized by a systolic blood pressure greater than 130 mmHg or a diastolic blood pressure greater than 80 mmHg without a single, distinct underlying cause.

As discussed in detail below, in some embodiments of the invention, the GLP-1/PYY, GIP and GLP-1/GIP compositions of the invention are adapted to effectively and safely (i) treat hypertension presented by a patient, i.e., reduce the systolic and/or diastolic blood pressure of the patient, (ii) ameliorate and/or stabilize at least one physiological risk factor associated with hypertension, and (iii) preferably, ameliorate and/or stabilize at least one pathophysiological effect associated with hypertension, when the GLP-1/PYY, GIP and GLP-1/GIP compositions are delivered to a patient.

As set forth in priority Co-Pending application Ser. No. 18/958,791, recent studies reflect that essential hypertension is associated with dysregulation of the renin-angiotensin-aldosterone system (RAAS), which is a core blood pressure regulation system that regulates angiotensin II (Ang II) and aldosterone, two seminal blood pressure-increasing endocrine factors. The studies have specifically found that dysregulation of the RAAS system results in increased levels of Ang II and aldosterone, which activate NADPH oxidase in vascular walls to increase concentrations of reactive oxygen species (ROS), such as O₂⁻ and H₂O₂, to induce vasoconstriction and, thereby, increase blood pressure to hypertensive levels.

The studies have also established that the excess levels of ROS will also activate the mitogen-activated protein kinase (MAPK) cell signaling pathway associated with endogenous cell proliferation, and the Smad-dependent transforming growth factor beta (TGF-β) cell signaling pathway associated with collagen synthesis, which results in maladaptive remodeling, e.g., thickening and fibrosis, of vascular walls and, thereby, vascular dysfunction and chronic hypertension. See Ashino, et al., Redox-Sensitive Transcription Factor Nrf2 Regulates Vascular Smooth Muscle Cell Migration and Neointimal Hyperplasia, Arteriosclerosis, Thrombosis, and Vascular Biology, vol. 33.4, pp. 760-768 (2013).

Recent studies also reflect that there is a direct relationship between insulin resistance and hypertension. See, e.g., Jia, et al., Hypertension in Diabetes: An Update of Basic Mechanisms and Clinical Disease, Hypertension, vol. 78.5, pp. 1197-1205 (2021).

As reflected in the studies, insulin resistance induces maladaptive activation of RAAS, which increases angiotensin II and aldosterone production and, thereby, activates NADPH oxidase in vascular walls and increases serine phosphorylation of insulin receptor substrate proteins, resulting in decreased activity of insulin downstream signaling pathways in phosphatidylinositide 3-kinase (PI3K) and protein kinase B (Akt) pathways, and leads to decreased endothelial nitric oxide synthase (eNOS) activation by insulin and reduced nitric oxide (NO) mediated vasodilation. The activation of NADPH oxidase and decreased nitric oxide (NO) mediated vasodilation promotes vasoconstriction of vascular walls and, thereby, promotes hypertension by increasing blood pressure.

The studies further reflect that insulin resistance also increases oxidative stress in vascular cells by increasing further activation of NADPH oxidases, which, as indicated above, increase concentrations of reactive oxygen species (ROS), such as O₂⁻ and H₂O₂, to induce vasoconstriction and, thereby, increase blood pressure to hypertensive levels.

The studies additionally reflect that insulin resistance also increases vascular inflammation by inducing TLR (Toll-like receptor)-mediated proinflammatory signaling, which induces activation of nuclear factor kappa B and c-Jun N-terminal kinase that promote release of proinflammatory cytokines from immune cells (such as macrophages), including tumor necrosis factor alpha (TNF-α), interleukin-6 (IL-6), vascular cell adhesion molecular 1 (VCAM1), and monocyte chemoattractant protein-1 (MCP-1). The proinflammatory cytokines induce seminal inflammatory processes that further impair insulin signaling and reduce insulin-mediated NO production, which promote vasoconstriction of vascular walls and, thereby, similarly promotes hypertension by increasing blood pressure.

According to the invention, when the GLP-1/PYY, GIP and GLP-1/GIP secretion compositions of the invention are delivered to a patient presenting with hypertension, the GLP-1/PYY, GIP and GLP-1/GIP secretion compositions effectively and safely treat the hypertension, i.e., reduce the systolic and/or diastolic blood pressure of the patient, and ameliorate and/or stabilize at least one physiological risk factor associated with hypertension and ameliorate and/or stabilize at least one pathophysiological effect associated with hypertension by (i) activating P13K and Akt signaling pathways and, thereby, increasing eNOS activation and NO presence in endothelial cells, (ii) attenuating MAPK cell signaling activity and, hence, maladaptive remodeling processes associated therewith, (iii) inducing sodium excretion, reducing H⁺ secretion, and reducing glomerular hyperfiltration and, thereby, attenuating maladaptive RAAS activation, and (iv) reducing vascular inflammation by decreasing TLR-mediated proinflammatory signaling, and (v) ameliorating apoptosis of myocardial cells (e.g., cardiomyocytes and cardiac fibroblasts) and vascular endothelial cells, myocardial inflammation, maladaptive myocardial remodeling (including myocardial fibrosis and associated myocardial ECM stiffening), maladaptive inflammatory gene expression.

Thus, according to the invention, in some embodiments, when the GLP-1/PYY, GIP and GLP-1/GIP secretion compositions of the invention are delivered to a patient presenting with hypertension, the GLP-1/PYY, GIP and GLP-1/GIP compositions of the invention can and will, effectively and safely treat hypertension presented by a patient by inducing multiple seminal physiological processes, including, without limitation, (i) increasing systemic insulin sensitivity, (ii) increasing eNOS activation, (ii) attenuating MAPK cell signaling activity and, hence, maladaptive remodeling processes associated therewith, (iii) inducing sodium excretion, reducing H⁺ secretion, and reducing glomerular hyperfiltration and, thereby, attenuating maladaptive RAAS activation, and (iv) reducing vascular inflammation by decreasing TLR-mediated proinflammatory signaling.

As indicated above, according to the invention, when the GLP-1/PYY, GIP and GLP-1/GIP secretion compositions of the invention are delivered to a patient presenting with hypertension, the noted GLP-1/PYY, GIP and GLP-1/GIP compositions will also ameliorate at least one physiological risk factor associated with hypertension, including, without limitation, systemic insulin resistance, impaired insulin production, diminished NO presence in the extracellular space proximate vascular endothelial cells, endothelial dysfunction, e.g., apoptosis of vascular endothelial and smooth muscle cells, vascular inflammation, maladaptive vascular remodeling (including vascular fibrosis and intimal neoplasia) and luminal stenosis.

Thus, in some embodiments of the invention, the GLP-1/PYY, GIP and GLP-1/GIP compositions of the invention, when delivered to a patient presenting with hypertension, can, and will, effectively and safely (i) treat the hypertension, i.e., reduce the systolic and/or diastolic blood pressure of the patient, (ii) ameliorate and/or stabilize at least one physiological risk factor associated with hypertension, and (iii) preferably ameliorate and/or stabilize at least one pathophysiological effect associated with hypertension.

Recent studies also reflect that there is a strong correlation between abnormal insulin secretion (and insulin resistance-associated) disorders; particularly, type 2 diabetes mellitus, and, hence, the physiological risk factors associated therewith; particularly, hyperglycemia, and hypertension. See, e.g., Petrie, et al., Diabetes, Hypertension, and Cardiovascular Disease: Clinical Insights and Vascular Mechanisms, Canadian Journal of Cardiology, vol. 34.5, pp. 575-584 (2018).

Indeed, as reflected in the studies, systemic insulin resistance and, hence, hyperglycemia associated therewith also induces increased activity of vasoconstrictor ET-1 (endothelin-1) via a mitogen-activated protein kinase-dependent signaling pathway and endothelial/epithelial sodium channels, which further increases vascular insulin resistance, induces increased arterial stiffening, and similarly promotes hypertension by increasing blood pressure.

The studies further confirm that insulin resistance and, hence, hyperglycemia associated glucotoxicity resulting therefrom, induces a protein glycation reaction resulting in increased production of advanced glycation end products (AGEs), which are produced from non-enzymatic glycosylation of lipids, lipoproteins and amino acids. Increased AGE production induces maladaptive vascular remodeling and, hence, changes in mechanical properties of the vascular extracellular matrix (ECM), e.g., increased vascular ECM stiffness and impaired vascular relaxation (and, hence, vasodilation), by increasing resistance to connective tissue enzymatic proteolysis and inducing the fibrotic crosslinking of the vascular ECM. The fibrotic crosslinking of the vascular ECM induces widening of arterial pulse pressure and increased pulsatile shear and, thereby, promotes vascular dysfunction, chronic vasoconstriction and, hence, an attendant hypertensive increase in blood pressure.

The AGEs also bind to the vascular cell (e.g., endothelial cells and smooth muscle cells) surface receptor for AGE (RAGE) to (i) induce maladaptive inflammatory gene expression, (ii) increase vascular ECM protein production via processes mediated via through mitogen-activated protein kinase (MAPK) and Janus kinase (JAK) pathways in the vascular system and (iii) increase the production of pro-inflammatory reactive oxygen species (ROS), which promote further fibrotic crosslinking of the vascular ECM and calcification of the vascular system.

In some instances, the binding of AGEs to the RAGE receptor of damaged endothelial and smooth muscle cells also induces apoptosis of damaged cells and, hence, a pro-inflammatory reaction to the apoptosis of the cells and the pro-apoptotic (and pro-inflammatory) cytokines released thereby that promotes further fibrotic crosslinking of the vascular ECM.

There is, thus, a strong correlation between the physiological risk factors associated with diabetes; particularly, type 2 diabetes mellitus, and hypertension.

Recent studies also reflect that there is a strong correlation between obesity and hypertension. See, e.g., Petrie, et al., Diabetes, Hypertension, and Cardiovascular Disease: Clinical Insights and Vascular Mechanisms, supra.

Indeed, as reflected in the studies, individuals with obesity typically have an excess supply of perivascular adipose tissue, which produces proinflammatory adipokines (e.g., leptin and aldosterone) and NADPH oxidases that, in combination, promote further systemic and vascular insulin resistance and inflammation, and induce impaired vascular relaxation and stiffness and, hence, similarly promotes a hypertensive increase in blood pressure.

As indicated above, in some embodiments of the invention, when the GLP-1/PYY, GIP and GLP-1/GIP compositions of the invention are delivered to a patient presenting with hypertension, the GLP-1/PYY, GIP and GLP-1/GIP compositions of the invention can, and will, effectively and safely treat the hypertension, and ameliorate and/or stabilize at least one physiological risk factor associated with hypertension and at least one pathophysiological effect associated with hypertension.

In some embodiments, if the patient also presents with insulin resistance of the vascular system, the GLP-1/PYY, GIP and GLP-1/GIP compositions of the invention will also effectively and safely treat the insulin resistance of the vascular system.

As indicated above and set forth in Applicant's priority U.S. application Ser. No. 18/980,129, when the GLP-1/PYY, GIP and GLP-1/GIP compositions of the invention are delivered to a patient presenting with obesity and/or type 2 diabetes mellitus, the GLP-1/PYY, GIP and GLP-1/GIP compositions of the invention can, and will, also effectively and safely treat the obesity, and ameliorate at least one pathophysiological effect associated with the obesity, and/or treat the type 2 diabetes mellitus, and ameliorate at least one physiological risk factor associated with type 2 diabetes mellitus and/or at least one pathophysiological effect associated with the type 2 diabetes mellitus.

Thus, in some embodiments of the invention, when the GLP-1/PYY, GIP and GLP-1/GIP secretion compositions of the invention are delivered to a patient presenting with hypertension, the GLP-1/PYY, GIP and GLP-1/GIP compositions can, and will, effectively and safely treat hypertension, and ameliorate at least one physiological risk factor associated with hypertension and/or at least one seminal pathophysiological effect associated with hypertension, and, if the patient also presents with obesity or type 2 diabetes mellitus or inflammation of the vascular system, the GLP-1/PYY, GIP and GLP-1/GIP compositions will also treat the obesity, and ameliorate at least one pathophysiological effect associated with the obesity, treat the type 2 diabetes mellitus, and ameliorate at least one physiological risk factor associated with type 2 diabetes mellitus and/or at least one pathophysiological effect associated with the type 2 diabetes mellitus, and treat the insulin resistance of the vascular system.

According to the invention, the noted GLP-1/PYY, GIP and GLP-1/GIP compositions similarly preferably comprise at least one of the natural compounds or ligands referenced above that is adapted to bind to and activate at least one olfactory receptor; specifically, olfactory receptor OR51E1, olfactory receptor OR1A1 or olfactory receptor OR2C1, and at least one free fatty acid receptor; specifically, free fatty acid receptor FFAR1 or FFAR4, and, in some instances, at least one transient receptor; specifically, transient receptor TRPA1.

In some embodiments, the noted GLP-1/PYY, GIP and GLP-1/GIP compositions similarly preferably comprise at least one of the natural compounds or ligands referenced above that is specifically adapted to bind to and activate at least olfactory receptor OR51E1 and free fatty acid receptor FFAR 1 or FFAR4.

According to the invention, the noted GLP-1/PYY, GIP and GLP-1/GIP secretion compositions can also comprise at least one natural compound or ligand that is adapted to bind to and activate hydroxycarboxylic acid receptor 2 (GPR109A) of endogenous cells, such as, without limitation, butyrate and nicotinic acid, whereby, when the noted GLP-1/PYY, GIP and GLP-1/GIP secretion compositions are delivered to a patient presenting with hypertension, the noted GLP-1/PYY, GIP and GLP-1/GIP secretion compositions attenuate the production and release of NADPH Oxidases (NOXs), nuclear factor κB (NK-κB) and renin by the cells and, hence, further facilitate treatment of the hypertension, and amelioration of at least one physiological risk factor associated with hypertension and/or at least one seminal pathophysiological effect associated with hypertension.

According to the invention, the noted GLP-1/PYY, GIP and GLP-1/GIP secretion compositions can also comprise at least one natural compound or ligand that is adapted to bind to and activate olfactory receptor OR51E2 of various endogenous cells, such as, without limitation, β-ionone, propionic acid and acetate, whereby, when the noted GLP-1/PYY, GIP and GLP-1/GIP secretion compositions are delivered to a patient presenting with hypertension, the noted GLP-1/PYY, GIP and GLP-1/GIP secretion compositions induce the release of endocrine factors, e.g., 5-HT, secretin, prostaglandin E₂ and vasoactive intestinal protein, from endogenous cells that induce activation of cell signaling pathways in vivo and, hence, further facilitate treatment of the hypertension, and amelioration of at least one physiological risk factor associated with hypertension and/or at least one seminal pathophysiological effect associated with hypertension.

According to the invention, the noted GLP-1/PYY, GIP and GLP-1/GIP secretion compositions will also activate olfactory receptor OR51E2 of endogenous smooth muscle cells of the small resistance blood vessels of the cardiovascular system, which will similarly further facilitate treatment of the hypertension, and amelioration of at least one physiological risk factor associated with hypertension and/or at least one seminal pathophysiological effect associated with hypertension.

As indicated above, in some embodiments of the invention, the GLP-1/PYY, GIP and GLP-1/GIP compositions comprise at least one of the natural compounds or ligands referenced above that is adapted to bind to and activate transient receptor TRPA1, whereby serotonin (5-HT) secretion from endogenous enterochromaffin (EC) cells is induced.

Recent studies reflect that, when TRPA1 is activated and serotonin secretion from EC cells is induced, the serotonin will bind to and activate 5-HT₁ receptors (such as 5-HT_{1A}, 5-HT_{1B}, and 5-HT_{1C} receptors), which induces smooth muscle relaxation by, among other seminal physiological activities, activating endothelial nitric oxide synthase (eNOS) and inducing nitric oxide (NO) mediated vasodilation, whereby effective treatment of hypertension is provided. See Mohammad-Zadeh, et al., Serotonin: A Review, supra.

As indicated above, preferred natural compound and ligands of the GLP-1/PYY, GIP and GLP-1/GIP compositions, which are adapted to activate transient receptor TRPA1 in vivo comprise allyl isothiocyanate, cinnamaldehyde, farnesyl thiosalicylic acid, formalin, hydrogen peroxide, 4-hydroxynonenal and acrolein.

In a preferred embodiment, the GLP-1/PYY, GIP and GLP-1/GIP compositions referenced above, wherein the compositions comprise a natural compound or ligand that is adapted to bind to and activate transient receptor TRPA1, also comprise at least one natural compound or ligand that is adapted to bind to and antagonize, i.e. restrict activity of, at least one 5-HT₂ receptor (such as 5-HT_{2A}, 5-HT_{2B} and 5-HT_{2C}) in vivo.

Recent studies reflect that, when at least one 5-HT₂ receptor expressed by platelet cells is antagonized, seminal 5-HT₂ receptor-induced cell signaling processes are abated, including inducement of the Gα_{q} signal transduction pathway. In addition to preventing atherosclerotic platelet aggregation, the abated inducement of seminal 5-HT₂ receptor-induce cell signaling processes will also prevent vasospasm-associated vasoconstriction, whereby hypertension is similarly effectively treated. See Marcinkowska, et al., Exploring the Antiplatelet Activity of Serotonin 5-HT2A Receptor Antagonists Bearing 6-Fluorobenzo [D] Isoxazol-3-Yl) Propyl) Motif-As Potential Therapeutic Agents in the Prevention of Cardiovascular Diseases Biomedicine & Pharmacotherapy, supra.

As indicated above, suitable natural compounds or ligands that are adapted to bind to and antagonize at least one 5-HT₂-serotonergic receptor, comprise, without limitation, the following natural antagonists: 3,3′,4′,5,6,7,8-heptamethoxyflavone (HMF), hesperidin and isoliquiritigenin (ISL).

Various exemplar and, hence, non-limiting, embodiments of GLP-1/PYY and GLP-1/GIP compositions of the invention that are adapted to directly treat hypertension, and ameliorate at least one physiological risk factor of hypertension and/or at least one seminal pathophysiological effect induced by the hypertension, and concurrently treat (i) hypertension, and ameliorate at least one physiological risk factor of hypertension and/or at least one seminal pathophysiological effect induced by the hypertension, and (ii) treat type 2 diabetes mellitus, and ameliorate at least one physiological risk factor of the type 2 diabetes mellitus and/or at least one seminal pathophysiological effect induced by the type 2 diabetes mellitus, and (iii) in some instances, treat insulin resistance of the vascular system, are set forth below.

In one embodiment of the invention, a GLP-1/PYY secretion composition for treating hypertension presented by a patient comprises at least one receptor activating compound adapted to bind to and activate at least one receptor selected from the group comprising olfactory receptor OR51E1, olfactory receptor OR1A1, olfactory receptor OR2C1 and olfactory receptor OR10J5,
the GLP-1/PYY secretion composition adapted to induce GLP-1 and PYY secretion, and, thereby, increased insulin secretion in vivo, whereby the hypertension is treated when the GLP-1/PYY secretion composition is delivered to a patient.

In a preferred embodiment, the GLP-1/PYY secretion composition is further adapted to ameliorate at least one at least one risk factor associated with the hypertension when the GLP-1/PYY secretion composition is delivered to the patient.

In some embodiments, the GLP-1/PYY secretion composition is further adapted to ameliorate at least one seminal pathophysiological effect associated with the hypertension when the GLP-1/PYY secretion composition is delivered to the patient.

In some embodiments, the receptor activating compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 0.5 μM to approximately 20.0 μM.

In some embodiments, the receptor activating compound comprises isovaleric acid.

In some embodiments, the isovaleric acid comprises an EC₅₀ value of at least approximately 0.1 μM in the GLP-1/PYY secretion composition.

In some embodiments, the isovaleric acid comprises an EC₅₀ value in the GLP-1/GIP secretion composition in the range of approximately 1.0 μM to approximately 100.0 μM.

In a preferred embodiment, the receptor activating compound is adapted to induce at least 50% activation of at least olfactory receptor OR51E1 in vivo when the GLP-1/PYY secretion composition is delivered to the patient.

In another embodiment of the invention, a GLP-1/PYY secretion composition for treating hypertension presented by a patient comprises at least one receptor activating compound adapted to bind to and activate at least one receptor selected from the group comprising olfactory receptor OR51E1 and olfactory receptor OR1A1,
the GLP-1/PYY secretion composition adapted to induce GLP-1 and PYY secretion, and, thereby, increased insulin secretion in vivo, whereby the hypertension is treated when the GLP-1/PYY secretion composition is delivered to a patient.

In a preferred embodiment, the GLP-1/PYY secretion composition is further adapted to ameliorate at least one at least one risk factor associated with the hypertension when the GLP-1/PYY secretion composition is delivered to the patient.

In some embodiments, the GLP-1/PYY secretion composition is further adapted to ameliorate at least one seminal pathophysiological effect associated with the hypertension when the GLP-1/PYY secretion composition is delivered to the patient.

In some embodiments, the receptor activating compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 0.5 μM to approximately 20.0 μM.

In some embodiments, the receptor activating compound comprises isovaleric acid.

In some embodiments, the isovaleric acid comprises an EC₅₀ value of at least approximately 0.1 μM in the GLP-1/PYY secretion composition.

In some embodiments, the isovaleric acid comprises an EC₅₀ value in the GLP-1/GIP secretion composition in the range of approximately 1.0 μM to approximately 100.0 μM.

In some embodiments, the receptor activating compound comprises geraniol.

In some embodiments, the geraniol comprises an EC₅₀ value of at least approximately 0.1 μM in the GLP-1/PYY secretion composition.

In some embodiments, the geraniol comprises an EC₅₀ value in the GLP-1/GIP secretion composition in the range of approximately 1.0 μM to approximately 30.0 μM.

In a preferred embodiment, the receptor activating compound is adapted to induce at least 50% activation of at least olfactory receptor OR51E1 in vivo when the GLP-1/PYY secretion composition is delivered to the patient.

In another embodiment of the invention, a GLP-1/PYY secretion composition for treating hypertension presented by a patient comprises a plurality of receptor activating compounds adapted to bind to and activate at least olfactory receptor OR51E1 and olfactory receptor OR1A1, the GLP-1/PYY secretion composition adapted to induce GLP-1 and PYY secretion, and, thereby, increased insulin secretion in vivo, whereby the hypertension is treated when the GLP-1/PYY secretion composition is delivered to a patient.

In a preferred embodiment, the GLP-1/PYY secretion composition is further adapted to ameliorate at least one at least one risk factor associated with the hypertension when the GLP-1/PYY secretion composition is delivered to the patient.

In some embodiments, the GLP-1/PYY secretion composition is further adapted to ameliorate at least one seminal pathophysiological effect associated with the hypertension when the GLP-1/PYY secretion composition is delivered to the patient.

In some embodiments, the plurality of receptor activating compounds comprise eugenol and geraniol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 0.5 μM to approximately 20.0 μM.

In some embodiments, the geraniol comprises an EC₅₀ value of at least approximately 0.1 μM in the GLP-1/PYY secretion composition.

In some embodiments, the geraniol comprises an EC₅₀ value in the GLP-1/GIP secretion composition in the range of approximately 1.0 μM to approximately 30.0 μM.

In a preferred embodiment, at least one of the plurality of receptor activating compounds is adapted to induce at least 50% activation of at least olfactory receptor OR51E1 in vivo when the GLP-1/PYY secretion composition is delivered to the patient.

In another embodiment of the invention, a GLP-1/PYY secretion composition for treating hypertension and an insulin-resistance-induced physiological disorder presenting by a patient comprises a plurality of receptor activating compounds adapted to bind to and activate at least olfactory receptor OR51E1 and olfactory receptor OR1A1,
the GLP-1/PYY secretion composition similarly adapted to induce GLP-1 and PYY secretion, and, thereby, increased insulin secretion in vivo, whereby the hypertension and insulin-resistance-induced physiological disorder are treated when the GLP-1/PYY secretion composition is delivered to a patient.

In some embodiments, the insulin-resistance-induced physiological disorder comprises type 2 diabetes mellitus.

In a preferred embodiment, the plurality of receptor activating compounds comprise eugenol and geraniol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 0.5 μM to approximately 20.0 μM.

In some embodiments, the geraniol comprises an EC₅₀ value of at least approximately 0.1 μM in the GLP-1/PYY secretion composition.

In some embodiments, the geraniol comprises an EC₅₀ value in the GLP-1/GIP secretion composition in the range of approximately 1.0 μM to approximately 30.0 μM.

In a preferred embodiment, at least one of the receptor activating compounds is adapted to induce at least 50% activation of at least OR51E1 in vivo when the GLP-1/PYY secretion composition is delivered to the patient.

In another embodiment of the invention, a GLP-1/GIP secretion composition for treating hypertension presented by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5, and
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1,
the GLP-1/GIP secretion composition adapted to induce GLP-1 and GIP secretion, and, thereby, increased insulin secretion in vivo, whereby the hypertension is treated when the GLP-1/GIP secretion composition is delivered to a patient.

In a preferred embodiment, the GLP-1/GIP secretion composition is further adapted to ameliorate at least one risk factor associated with the hypertension when the GLP-1/GIP secretion composition is delivered to the patient.

In some embodiments, the GLP-1/GIP secretion composition is further adapted to ameliorate at least one seminal pathophysiological effect associated with the hypertension when the GLP-1/GIP secretion composition is delivered to the patient.

In some embodiments, the first receptor activating compound comprises a first compound selected from the group comprising eugenol, isovaleric acid and butyrate.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 0.5 μM to approximately 20.0 μM.

In some embodiments, the first compound comprises isovaleric acid.

In some embodiments, the isovaleric acid comprises an EC₅₀ value of at least approximately 0.1 μM in the GLP-1/PYY secretion composition.

In some embodiments, the isovaleric acid comprises an EC₅₀ value in the GLP-1/GIP secretion composition in the range of approximately 1.0 μM to approximately 100.0 μM.

In some embodiments, the first compound comprises butyrate.

In some embodiments, the butyrate comprises an EC₅₀ value of at least approximately 0.1 μM in the GLP-1/PYY secretion composition.

In some embodiments, the butyrate comprises an EC₅₀ value in the GLP-1/GIP secretion composition in the range of approximately 1.0 μM to approximately 200.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/PYY secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises cinnamaldehyde.

In some embodiments, the cinnamaldehyde comprises an EC₅₀ value of at least approximately 0.05 μM in the GLP-1/PYY secretion composition.

In some embodiments, the cinnamaldehyde comprises an EC₅₀ value in the GLP-1/GIP secretion composition in the range of approximately 0.1 μM to approximately 2500.0 μM.

In some embodiments, the second receptor activating compound comprises acetate.

In some embodiments, the acetate comprises an EC₅₀ value of at least approximately 0.05 μM in the GLP-1/PYY secretion composition.

In some embodiments, the acetate comprises an EC₅₀ value in the GLP-1/GIP secretion composition in the range of approximately 0.1 μM to approximately 200.0 μM.

In a preferred embodiment, at least one of the first receptor activating compounds is adapted to induce at least 50% activation of at least olfactory receptor OR51E1 in vivo when the GLP-1/GIP secretion composition is delivered to the patient.

In another embodiment of the invention, a GLP-1/GIP secretion composition for treating hypertension and an insulin-resistance-induced physiological disorder presenting by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5, and
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1,
the GLP-1/GIP secretion composition adapted to induce GLP-1 secretion, and, thereby, increased insulin secretion in vivo, whereby the hypertension and insulin-resistance-induced physiological disorder are treated when the GLP-1/PYY secretion composition is delivered to a patient.

In a preferred embodiment, the GLP-1/GIP secretion composition is further adapted to ameliorate at least one risk factor associated with the hypertension when the GLP-1/GIP secretion composition is delivered to the patient.

In some embodiments, the GLP-1/GIP secretion composition is further adapted to ameliorate at least one seminal pathophysiological effect associated with the hypertension when the GLP-1/GIP secretion composition is delivered to the patient.

In some embodiments, the insulin-resistance-induced physiological disorder comprises type 2 diabetes mellitus.

In some embodiments, the GLP-1/GIP secretion composition is further adapted to ameliorate at least one risk factor associated with the type 2 diabetes mellitus when the GLP-1/GIP secretion composition is delivered to the patient.

In some embodiments, the first receptor activating compound comprises a first compound selected from the group comprising eugenol, isovaleric acid and butyrate.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 0.5 μM to approximately 20.0 μM.

In some embodiments, the first compound comprises isovaleric acid.

In some embodiments, the isovaleric acid comprises an EC₅₀ value of at least approximately 0.1 μM in the GLP-1/PYY secretion composition.

In some embodiments, the isovaleric acid comprises an EC₅₀ value in the GLP-1/GIP secretion composition in the range of approximately 1.0 μM to approximately 100.0 μM.

In some embodiments, the first compound comprises butyrate.

In some embodiments, the butyrate comprises an EC₅₀ value of at least approximately 0.1 μM in the GLP-1/PYY secretion composition.

In some embodiments, the butyrate comprises an EC₅₀ value in the GLP-1/GIP secretion composition in the range of approximately 1.0 μM to approximately 200.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/PYY secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises cinnamaldehyde.

In some embodiments, the cinnamaldehyde comprises an EC₅₀ value of at least approximately 0.05 μM in the GLP-1/PYY secretion composition.

In some embodiments, the cinnamaldehyde comprises an EC₅₀ value in the GLP-1/GIP secretion composition in the range of approximately 0.1 μM to approximately 2500.0 μM.

In some embodiments, the second receptor activating compound comprises acetate.

In some embodiments, the acetate comprises an EC₅₀ value of at least approximately 0.05 μM in the GLP-1/PYY secretion composition.

In some embodiments, the acetate comprises an EC₅₀ value in the GLP-1/GIP secretion composition in the range of approximately 0.1 μM to approximately 200.0 μM.

In a preferred embodiment, at least one of the first receptor activating compounds is adapted to induce at least 50% activation of at least olfactory receptor OR51E1 in vivo when the GLP-1/GIP secretion composition is delivered to the patient.

In another embodiment of the invention, a GLP-1/GIP secretion composition for treating hypertension presented by a patient comprises:
(i) at least a first receptor activating compound adapted to bind to and activate at least one first receptor selected from the group comprising olfactory receptor OR51E1 and olfactory receptor OR1A1,
(ii) at least a second receptor activating compound adapted to bind to and activate at least one second receptor selected from the group comprising free fatty acid receptor FFAR1 and free fatty acid receptor FFAR4, and
(iii) at least a third receptor activating compound adapted to bind to and activate olfactory receptor OR51E2,
the GLP-1/GIP secretion composition adapted to induce GLP-1 and GIP secretion, and, thereby, increased insulin secretion in vivo, whereby the hypertension is treated when the GLP-1/GIP secretion composition is delivered to a patient.

In a preferred embodiment, the GLP-1/GIP secretion composition is further adapted to ameliorate at least one risk factor associated with the hypertension when the GLP-1/GIP secretion composition is delivered to the patient.

In some embodiments, the GLP-1/GIP secretion composition is further adapted to ameliorate at least one seminal pathophysiological effect associated with the hypertension when the GLP-1/GIP secretion composition is delivered to the patient.

In some embodiments, the first receptor activating compound comprises a first compound selected from the group comprising eugenol, isovaleric acid and butyrate.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 0.5 μM to approximately 20.0 μM.

In some embodiments, the first compound comprises isovaleric acid.

In some embodiments, the isovaleric acid comprises an EC₅₀ value of at least approximately 0.1 μM in the GLP-1/PYY secretion composition.

In some embodiments, the isovaleric acid comprises an EC₅₀ value in the GLP-1/GIP secretion composition in the range of approximately 1.0 μM to approximately 100.0 μM.

In some embodiments, the first compound comprises butyrate.

In some embodiments, the butyrate comprises an EC₅₀ value of at least approximately 0.1 μM in the GLP-1/PYY secretion composition.

In some embodiments, the butyrate comprises an EC₅₀ value in the GLP-1/GIP secretion composition in the range of approximately 1.0 μM to approximately 200.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/PYY secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises acetate.

In some embodiments, the acetate comprises an EC₅₀ value of at least approximately 0.05 μM in the GLP-1/PYY secretion composition.

In some embodiments, the acetate comprises an EC₅₀ value in the GLP-1/GIP secretion composition in the range of approximately 0.1 μM to approximately 200.0 μM.

In some embodiments, the third receptor activating compound comprises a third compound selected from the group comprising β-ionone and propionic acid.

In some embodiments, the third compound comprises β-ionone.

In some embodiments, the β-ionone comprises an EC₅₀ value of at least approximately 0.1 μM in the GLP-1/GIP secretion composition.

In some embodiments, the β-ionone comprises an EC₅₀ value in the GLP-1/GIP secretion composition in the range of approximately 5.0 μM to approximately 250.0 μM.

In a preferred embodiment, at least one of the first receptor activating compounds is adapted to induce at least 50% activation of at least olfactory receptor olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In a preferred embodiment, at least one of the third receptor activating compounds is adapted to induce at least 50% activation of olfactory receptor OR51E2 in vivo when the GLP-1/GIP secretion composition is delivered to the patient.

In another embodiment of the invention, a GLP-1/GIP secretion composition for treating hypertension presented by a patient comprises:
(i) at least a first receptor activating compound adapted to bind to and activate at least one first receptor selected from the group comprising olfactory receptor OR51E1 and olfactory receptor OR1A1,
(ii) at least a second receptor activating compound adapted to bind to and activate at least one second receptor selected from the group comprising free fatty acid receptor FFAR1 and free fatty acid receptor FFAR4, and
(iii) at least a second receptor activating compound adapted to bind to and activate olfactory receptor OR2L13,
the GLP-1/GIP secretion composition adapted to induce GLP-1 and GIP secretion, and, thereby, increased insulin secretion in vivo, whereby the hypertension is treated when the GLP-1/GIP secretion composition is delivered to a patient.

In a preferred embodiment, the GLP-1/GIP secretion composition is further adapted to ameliorate at least one risk factor associated with the hypertension when the GLP-1/GIP secretion composition is delivered to the patient.

In some embodiments, the GLP-1/GIP secretion composition is further adapted to ameliorate at least one seminal pathophysiological effect associated with the hypertension when the GLP-1/GIP secretion composition is delivered to the patient.

In some embodiments, the first receptor activating compound comprises a first compound selected from the group comprising eugenol, isovaleric acid and butyrate.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 0.5 μM to approximately 20.0 μM.

In some embodiments, the first compound comprises isovaleric acid.

In some embodiments, the isovaleric acid comprises an EC₅₀ value of at least approximately 0.1 μM in the GLP-1/PYY secretion composition.

In some embodiments, the isovaleric acid comprises an EC₅₀ value in the GLP-1/GIP secretion composition in the range of approximately 1.0 μM to approximately 100.0 μM.

In some embodiments, the first compound comprises butyrate.

In some embodiments, the butyrate comprises an EC₅₀ value of at least approximately 0.1 μM in the GLP-1/PYY secretion composition.

In some embodiments, the butyrate comprises an EC₅₀ value in the GLP-1/GIP secretion composition in the range of approximately 1.0 μM to approximately 200.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/PYY secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises acetate.

In some embodiments, the acetate comprises an EC₅₀ value of at least approximately 0.05 μM in the GLP-1/PYY secretion composition.

In some embodiments, the acetate comprises an EC₅₀ value in the GLP-1/GIP secretion composition in the range of approximately 0.1 μM to approximately 200.0 μM.

In some embodiments, the third receptor activating compound comprises a third compound selected from the group comprising (−)-carvone and choline.

In some embodiments, the third compound comprises (−)-carvone.

In some embodiments, the (−)-carvone comprises an EC₅₀ value of at least approximately 0.1 μM in the GLP-1/GIP secretion composition.

In some embodiments, the (−)-carvone comprises an EC₅₀ value in the GLP-1/GIP secretion composition in the range of approximately 0.01 μM to approximately 100.0 μM.

In a preferred embodiment, at least one of the first receptor activating compounds is adapted to induce at least 50% activation of at least olfactory receptor OR51E1 in vivo when the GLP-1/GIP secretion composition is delivered to the patient.

In a preferred embodiment, the third receptor activating compound is adapted to induce at least 50% activation of olfactory receptor OR2L13 in vivo when the GLP-1/GIP secretion composition is delivered to the patient.

In another embodiment of the invention, a GLP-1/GIP secretion composition for treating hypertension presented by a patient comprises:
(i) at least a first receptor activating compound adapted to bind to and activate at least one first receptor selected from the group comprising olfactory receptor OR51E1, olfactory receptor OR1A1 and olfactory receptor OR2C1, and
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1,
the GLP-1/GIP secretion composition adapted to induce GLP-1 secretion, and, thereby, increased insulin secretion in vivo, whereby the hypertension is treated when the GLP-1/PYY secretion composition is delivered to a patient.

In a preferred embodiment, the GLP-1/GIP secretion composition is further adapted to ameliorate at least one risk factor associated with the hypertension when the GLP-1/GIP secretion composition is delivered to the patient.

In some embodiments, the GLP-1/GIP secretion composition is further adapted to ameliorate at least one seminal pathophysiological effect associated with the hypertension when the GLP-1/GIP secretion composition is delivered to the patient.

In some embodiments, the first receptor activating compound comprises a first compound selected from the group comprising eugenol, isovaleric acid and butyrate.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 0.5 μM to approximately 20.0 μM.

In some embodiments, the first compound comprises isovaleric acid.

In some embodiments, the isovaleric acid comprises an EC₅₀ value of at least approximately 0.1 μM in the GLP-1/PYY secretion composition.

In some embodiments, the isovaleric acid comprises an EC₅₀ value in the GLP-1/GIP secretion composition in the range of approximately 1.0 μM to approximately 100.0 μM.

In some embodiments, the first compound comprises butyrate.

In some embodiments, the butyrate comprises an EC₅₀ value of at least approximately 0.1 μM in the GLP-1/PYY secretion composition.

In some embodiments, the butyrate comprises an EC₅₀ value in the GLP-1/GIP secretion composition in the range of approximately 1.0 μM to approximately 200.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/PYY secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising acetate and cinnamaldehyde.

In some embodiments, the third compound comprises acetate.

In some embodiments, the acetate comprises an EC₅₀ value of at least approximately 0.05 μM in the GLP-1/PYY secretion composition.

In some embodiments, the acetate comprises an EC₅₀ value in the GLP-1/GIP secretion composition in the range of approximately 0.1 μM to approximately 200.0 μM.

In some embodiments, the third compound comprises cinnamaldehyde.

In some embodiments, the cinnamaldehyde comprises an EC₅₀ value of at least approximately 0.05 μM in the GLP-1/PYY secretion composition.

In some embodiments, the cinnamaldehyde comprises an EC₅₀ value in the GLP-1/GIP secretion composition in the range of approximately 0.1 μM to approximately 2500.0 μM.

In some embodiments, the composition further comprises at least a third receptor activating compound adapted to bind to and restrict 5-HT₂ serotonergic receptor activity.

In some embodiments, the third receptor activating compound comprises a fourth compound selected from the group comprising 3,3′,4′,5,6,7,8-heptamethoxyflavone (HMF), hesperidin and isoliquiritigenin (ISL).

In some embodiments, the fourth compound comprises HMF.

In some embodiments, the HMF comprises an EC₅₀ value of at least approximately 0.01 μM in the composition.

As indicated above, in some embodiments of the invention, there are thus also provided methods for treating a cardiovascular disorder presented by a patient.

In some embodiments, a method for treating a cardiovascular disorder presented by a patient comprises the steps of:
(a) providing one of the GLP-1/PYY or GLP-1/GIP secretion compositions of the invention; and
(b) delivering the composition to the patient, wherein insulin secretion of the patient is modulated in vivo, whereby the cardiovascular disorder is effectively treated.

In some embodiments, a method for treating a cardiovascular disorder presented by a patient comprises the steps of:
(a) providing one of the GLP-1/PYY or GLP-1/GIP secretion compositions of the invention; and
(b) delivering the composition to the patient, wherein insulin secretion of the patient is modulated in vivo, whereby the cardiovascular disorder is effectively treated.

In some embodiments, a method for treating a cardiovascular disorder presented by a patient comprises the steps of:
(a) providing one of the GLP-1/PYY or GLP-1/GIP secretion compositions of the invention; and
(b) delivering the composition to the patient, wherein insulin secretion and 5-HT₂-serotongeric receptor activity of the patient is modulated in vivo, whereby the cardiovascular disorder is effectively treated.

In some embodiments of the invention, there are also provided methods for treating a cardiovascular disorder and an insulin-resistance-induced physiological disorder presented by a patient.

In some embodiments, a method for treating a cardiovascular disorder and an insulin-resistance-induced physiological disorder presented by a patient comprises the steps of:
(a) providing one of the GLP-1/PYY or GLP-1/GIP secretion compositions of the invention; and
(b) delivering the composition to the patient, wherein insulin secretion of the patient is modulated in vivo, whereby the cardiovascular disorder and insulin-resistance-induced physiological disorder are treated.

As will readily be appreciated by one having ordinary skill in the art, the present invention provides numerous advantages compared to prior art formulations and methods for enhancing cell function and, thereby, mental function and acuity. Among the advantages are the following:
The provision of natural compounds and ligands, and compositions comprising same, for treating physiological disorders; particularly, insulin-resistance-induced physiological disorders, such as type 2 diabetes mellitus, and cardiovascular disorders, which substantially reduce or overcome the drawbacks and disadvantages associated with administration (or delivery) of GLP-1 analogs and dual GLP-1/GIP analogs to a patient presenting with a physiological disorder;
The provision of natural compounds and ligands, and compositions comprising same, which, when administered to a patient presenting with a physiological disorder, effectively treat the physiological disorder by modulating ectopic olfactory receptor activity;
The provision of natural compounds and ligands, and compositions comprising same, which effectuate olfactory receptor (OR)-mediated secretion of endogenous GLP-1 and PYY in a patient presenting with a physiological disorder, without the undesirable side effects associated with delivery of a GLP-1 analog and/or a dual GLP-1/GIP analog to patients;
The provision of natural compounds and ligands, and compositions comprising same, which effectuate OR-mediated, free fatty acid receptor-mediated, and transient potential ion channel-mediated secretion of endogenous GLP-1, PYY and GIP in a subject presenting with a physiological disorder, without the undesirable side effects associated with delivery of a GLP-1 analog and/or a dual GLP-1/GIP analog to patients;
The provision of natural compounds and ligands, and compositions comprising same, which, when administered to a patient, effectively and safely modulate the patient's systemic insulin secretion in vivo, and can be administered to the patient via oral, sublingual, inhalation, intranasal, epidural, intracerebral, transdermal, topical, and injection administration means;
The provision of natural compounds and ligands, and compositions comprising same, which, when administered to a patient presenting with an insulin-resistance-induced physiological disorder; particularly, type 2 diabetes mellitus, effectively and safely modulate the patient's insulin secretion in vivo, whereby the insulin-resistance-induced physiological disorder is effectively treated and at least one risk factor associated with the insulin-resistance-induced physiological disorder is ameliorated with minimal, if any, adverse side effects;
The provision of natural compounds and ligands, and compositions comprising same, which, when administered to a patient presenting with a cardiovascular disorder, effectively and safely modulate the patient's insulin secretion in vivo, whereby the cardiovascular disorder is effectively treated and at least one risk factor associated with the cardiovascular disorder is ameliorated with minimal, if any, adverse side effects;
The provision of natural compounds and ligands, and compositions comprising same, which, when administered to a patient presenting with a cardiovascular disorder, effectively and safely modulate the patient's insulin secretion and restrict 5-HT₂ serotonergic receptor activity in vivo, whereby the cardiovascular disorder is effectively treated and at least one risk factor associated with the cardiovascular disorder is ameliorated with minimal, if any, adverse side effects; and
The provision of natural compounds and ligands, and compositions comprising same, which, when administered to a patient presenting with a cardiovascular disorder and an insulin resistance-induced physiological disorder; particularly, type 2 diabetes mellitus, effectively and safely modulate the patient's insulin secretion in vivo, whereby the cardiovascular disorder and/or insulin-resistance-induced physiological disorder is effectively treated and at least one risk factor associated with the cardiovascular disorder or insulin-resistance-induced physiological disorder is ameliorated with minimal, if any, adverse side effects.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A composition for treating atherosclerosis presented by a patient, comprising:
(i) at least a first receptor activating compound adapted to bind to and activate at least one first receptor selected from the group comprising olfactory receptor family 51 subfamily E member 1 OR51E1) and olfactory receptor family 1 subfamily A member 1 (OR1A1),
(ii) at least a second receptor activating compound adapted to bind to and activate at least one second receptor selected from the group comprising free fatty acid receptor 1 (FFAR1) and free fatty acid receptor 4(FFAR4), and
(iii) at least a third receptor activating compound adapted to bind to and activate olfactory receptor family 2 subfamily L member 13 (OR2L13),
said composition adapted to induce GLP-1 secretion, and, thereby, increased insulin secretion in vivo, whereby said atherosclerosis is treated when said composition is delivered to said patient.

2. The composition of claim 1, wherein said composition is further adapted to ameliorate at least one risk factor of said atherosclerosis when said composition is said delivered to said patient.

3. The composition of claim 1, wherein said composition is further adapted to ameliorate at least one pathophysiological effect induced by said atherosclerosis when said composition is said delivered to said patient.

4. The composition of claim 1, wherein said first receptor activating compound is selected from the group consisting of eugenol and 3-methylpentanoic acid.

5. The composition of claim 4, wherein said first receptor activating compound comprises said eugenol.

6. The composition of claim 5, wherein said eugenol comprises a first EC₅₀ concentration in said composition of at least 0.1 μM.

7. The composition of claim 6, wherein said first EC₅₀ concentration of said eugenol in said composition is in the range from about 1.0 μM to about 15.0 μM.

8-10. (canceled)

11. The composition of claim 1, wherein said second receptor activating compound is selected from the group consisting of a medium-chain free fatty acid, a long-chain free fatty acid and an omega-3 polyunsaturated fatty acid.

12. The composition of claim 11, wherein said second receptor activating compound comprises a medium-chain free fatty acid.

13. The composition of claim 11, wherein said medium-chain free fatty acid comprises lauric acid.

14. The composition of claim 13, wherein said lauric acid comprises a second EC₅₀ concentration in said composition of at least 0.05 μM.

15. The composition of claim 14, wherein said second EC₅₀ concentration of said lauric acid in said composition is in the range from about 0.05 μM to about 50.0 μM.

16. The composition of claim 1, wherein said third receptor activating compound is selected from the group consisting of (−)-carvone and choline.

17. The composition of claim 16, wherein said third receptor activating compound comprises said (−)-carvone.

18. The composition of claim 17, wherein said (−)-carvone comprises a third EC₅₀ concentration in said composition of at least 0.1 μM.

19. The composition of claim 18, wherein said third EC₅₀ concentration of said (−)-carvone in said composition is in the range from about 0.01 μM to about 100.0 μM.

20. The composition of claim 1, wherein said composition further comprises at least a fourth receptor activating compound adapted to bind to and restrict 5-HT₂ serotonergic receptor activity.

21. The composition of claim 20, wherein said fourth receptor activating compound is selected from the group consisting of 3,3′,4′,5,6,7,8-heptamethoxyflavone (HMF), hesperidin and isoliquiritigenin (ISL).

22. The composition of claim 21, wherein said fourth receptor activating compound comprises said HMF.

23. The composition of claim 22, wherein said HMF comprises a fourth EC₅₀ concentration in said composition of at least approximately 0.01 μM.

24. The composition of claim 23, wherein said fourth EC₅₀ concentration of said HMF in said composition is in the range from about 0.01 μM to about 100.0 μM.

25. The composition of claim 1, wherein said first receptor activating compound is adapted to induce at least 50% activation of at least said olfactory receptor OR51E1 in vivo when said composition is said delivered to said patient.
